(19) United States
(12) Patent Application Publication
(10) Pub. No.: US 2015/0086981 A1
(43) Pub. Date: Mar. 26, 2015

(54) NUCLEOSIDE-TRIPHOSPHATE CONJUGATE AND METHODS FOR THE USE THEREOF

(71) Applicant: GENOVOXX GMBH, Luebeck (DE)

(72) Inventors: Dmitry Cherkasov, Marburg (DE); Claus Becker, Otigheim (DE); Norbert Basler, Gross Hansdorf (DE); Andreas Müller-Hermann, Munchen (DE); Petra Van Husen, Eltville (DE)

(73) Assignee: GENOVOXX GMBH, Luebeck (DE)

(21) Appl. No.: 14/115,564

(22) PCT Filed: May 4, 2012
(86) PCT No.: PCT/EP2012/001911

§ 371 Date: Oct. 17, 2014

(30) Foreign Application Priority Data
May 4, 2011 (DE) 10 2011 100 496.7

(51) Int. Cl. C12Q 1/68

(52) U.S. Cl. CPC C12Q 1/6876
USPC 435/6.11; 536/24.3

(57) ABSTRACT

The invention relates to a novel method for the enzymatic marking of nucleic acid chains (target sequences) with nucleotide conjugates. Under reaction conditions, said nucleotide conjugates are able to bind to a target sequence, and can be incorporated into the complementary growing strand by way of a polymerase. The nucleotide conjugates can be used for sequencing nucleic acid chains.

### DESCRIPTION OF THE INVENTION

### Introduction

### 1.1 State of the Art and Objects of the Invention

Powerful sequencing methods are known in modern biotechnology (“second generation” sequencing technologies, such as Illumina's Solexa technology). These methods are based on sequencing by synthesis and employ reversible terminators. In the context of developing procedures for sequencing by synthesis it is important to provide novel reversibly terminating nucleotide modifications. Better signal properties play a major role in sequencing, particularly at the single molecule level.

### 1.2 Object of the Invention

In one advantageous embodiment, the present invention describes new structures of nucleotide conjugates as well as methods for their application. Such nucleotide conjugates can be used in nucleic acid labeling reactions or in a sequencing reaction. In one advantageous embodiment, such conjugates may be used as reversible terminators in a sequencing-by-synthesis method.

Novel nuc-macromolecules with new marker component structures and new functions are provided. The nucleotide structures represent new nuc-macromolecule compositions of the basic structure described in applications Cherkasov et al WO2011050938, Cherkasov et al WO2005044836, Cherkasov et al WO2006097320, Cherkasov et al WO2008043426, Cherkasov et al DE 10356837, Cherkasov et al DE 102004009704. These applications are incorporated herein by reference in full scope. Nuc-macromolecules comprise at least one nuc-component, a 2′-deoxynucleoside-triphosphate for instance, at least one macromolecular marker and at least one linker between this marker and the nuc-component.

In one advantageous embodiment of the invention, nucleotide conjugates are described which comprise at least one nucleoside triphosphate, at least one oligonucleotide, as well as a linker between the nucleoside triphosphate and the oligonucleotide. The oligonucleotide in such a nucleotide conjugate is preferably part of the marker.

In one embodiment, the coupling of the linker to the nucleoside triphosphate (nuc-component of the nucleotide conjugate) is effected at the base (e.g. at the 5-position of pyrimidines or the 7-position of 7-deazapurines).

In another embodiment, the coupling of the linker is effected at the terminal phosphate group of the nucleotide (e.g. gamma-phosphate group in a nucleoside triphosphate).

In another embodiment, the coupling of the linker is effected at the 3′-position of the sugar of the nucleotide (e.g. at the 3′-OH group of a 2′-deoxyribose).

In a preferred embodiment, the linker contains at least one cleavable group, a disulfide group for example.

In one embodiment, the coupling of the linker to the oligonucleotide is effected at the 5′-end of the oligonucleotide. In another embodiment, the coupling of the linker to the oligonucleotide is effected at the 3′-end of the oligonucleotide. In another embodiment, the coupling of the linker to the oligonucleotide is effected at an internal position of the oligonucleotide.

In one embodiment of the invention, the composition of the oligonucleotide is chosen in such a way that it cannot bind to nucleic acid sequences to be labeled. This can be achieved, for example, by means of complete or partial double strand formation within the oligonucleotide (e.g. hairpin structures), or through the choice of appropriate reaction conditions.

In a further embodiment of the invention, the composition of the oligonucleotide is chosen such that it is able to bind to at least one nucleic acid sequence to be labeled.

In one embodiment of the invention, a method for the synthesis of nucleic acid chains is described in which at least four types of nucleotide-conjugates (e.g. dATP-conjugate, dCTP-conjugate, dGTP-conjugate and dUTP-conjugate) are incubated simultaneously with at least one primer-template complex and at least one DNA polymerase under conditions that allow for incorporation of a complementary nucleoside triphosphate of the nucleotide conjugate into the primer.

Nuc-macromolecules that undergo predominantly or completely sequence-specific binding to a target sequence are described in Cherkasov et al. WO2011050938.

A further embodiment of the invention includes nucleotide conjugates that bind more strongly to nucleic acid chains that are to be labeled, wherein this binding is mostly not sequence-specific. Such nucleotide conjugates have the capacity for non-sequence-specific binding to a plurality of different nucleic acid chains that are to be labeled. Such non-sequence-specific binding of nucleotide conjugates opens up unexpected applications. Significantly lower concentrations of nucleotide conjugates may be used to achieve a nuc-macromolecule incorporation event for example. The use of low nucleotide conjugate concentrations is of advantage, for instance, in methods for single-molecule nucleic acid analysis. Low concentrations cause significantly less background signal. This can lead to an increase in the quality of the signals in an assay.

In one embodiment, this non-sequence-specific binding to nucleic acid chains is achieved for example via base pairing between the oligonucleotide of the nucleotide conjugate and the nucleic acid chain to be labeled, where such base pairing occurs only over a relatively short stretch of the nucleic acid chain (3-15 bases for example) and therefore preferably exhibits low sequence specificity. In this embodiment of the invention, the sequence of the oligonucleotide within the nucleotide conjugate comprises at least one sequence segment that can bind to the nucleic acid chains by base pairing. This region of the oligonucleotide sequence is preferably single-stranded. The length of this fragment is preferably chosen in such a manner that the oligonucleotide may bind to the nucleic acid chain to be labeled through formation of, for example, 3 to 6, 7 to 10, 10 to 15 base pairs (consecutive base pairing, or separated by non-complementary segments).

In a further embodiment, nucleotide conjugates are incubated together with nucleic acid chains under conditions that allow for non-sequence-specific binding interactions. By way of example, nucleotide conjugates with longer oligonucleotides (e.g. between 15 and 50 nucleotides) are capable of binding to nucleic acid chains with low sequence specificity under non-stringent conditions.

In a further embodiment of the invention, nucleotide conjugate structures include positively charged moieties, such as a poly lysine-chain or polyethyleneimine (PEI), which bind to the nucleic acid chains by virtue of electrostatic charge interactions.

These moieties can play the role of a linker between the nuc-component and the oligonucleotide component (e.g. 2-10 lysine residues as a short peptide). Peptide nucleic acids (PNA) that have a positively charged backbone may be used as oligonucleotides within the nuc-macromolecule for example.

In a further embodiment of the invention, nucleotide conjugate structures include proteins that are capable of non-sequence-specific binding to nucleic acid chains, e.g. a single-strand binding protein.

In the interest of demonstration, this application provides detailed descriptions of nucleotide conjugates that comprise at least one oligonucleotide for improved binding to a nucleic acid chain to be labeled.

In a further embodiment of the invention, at least one composition consisting of several nuc-macromolecules that possess an identical nuc-component is employed. Such a composition preferably includes an identical or uniform type of nuc-component, a dATP-analog for example, which is coupled to different oligonucleotides.

Each of the oligonucleotides in such a composition includes at least one sequence segment that is capable of binding to at least one nucleic acid chain to be labeled. This sequence segment is preferably single-stranded. The length of this segment is preferably chosen in such a way that each oligonucleotide can bind to the nucleic acid chain that is to be labeled under formation of 3 to 20 base pairs, but preferably of 3 to 10 base pairs, and more preferably of 3 to 6 base pairs.

Such sequence segments may also be referred to as the binding segments of the oligonucleotides. They are referred to as “B-segments”. Such B-segments vary among the oligonucleotides of one nucleotide conjugate population (Segment B(1), B(2), B(3) etc. until B(n)). In one embodiment of the invention, the composition of the B-segments within a population represents all possible permutations (e.g. randomized hexamer with 4̂n variants, wherein (n) represents the number of nucleotide monomers in an oligonucleotide). In a further embodiment of the invention, the composition of the B-segments within a population is limited to a smaller number of selected oligonucleotide variants, where the number of different oligonucleotide variants can range from 10 to 100,000.

Further, each of the oligonucleotides of such a composition contains one signal-generating or signal-transmitting marker that is characteristic for this composition, for example a dye or another sequence segment of the oligonucleotide that is uniform for all oligonucleotides.

In summary, a composition of nucleotide conjugates comprises a uniform nuc-component, e.g. a uniform nucleoside triphosphate, and a population-specific signal-generating or signal-transmitting marker, as well as a plurality of oligonucleotides. Oligonucleotides within a composition differ from one another in the structure of their B-segments. The total number of the variants of the oligonucleotides within such a composition ranges between 4̂3 and 4̂50, but more preferably between 4̂5 and 4̂20, and even more preferably between 4̂6 and 4̂15. The length of the oligonucleotides is chosen accordingly so that the desired number of variants can be achieved. For instance, when the length of the B-segment is 3 bases, the complexity of the population is 64 (=4̂3), the complexity of the population is 256 (=4̂4) when the length of the B-segment is 4 bases, the complexity of the population is 1024 (4̂5) when the length of the B-segment is 5 bases, the complexity of the population is 4096 (=4̂6) when the length of the B-segment is 6 bases etc. If one composition covers all possible base sequence combinations, then such a population of nucleotide conjugates can bind to single-stranded nucleic acid chains of any composition. Said composition of nucleotide conjugates is preferably incubated with a nucleic acid chain to be labeled under reaction conditions that allow for reversible binding between oligonucleotides and the nucleic acid chain. This can be controlled via the reaction temperature for example. Under suitable temperature conditions, binding takes place between oligonucleotides of the nuc-macromolecules and single strands of the nucleic acid chains that are to be labeled, resulting in nucleotide conjugate-template complexes.

In one embodiment, the reaction temperature is lower than the Tm of potential nucleotide conjugate-template complexes (e.g. Tm minus 5° C.). The formation of nucleotide conjugate-template complexes is favored under such conditions. In a further embodiment, the reaction temperature is near the Tm of potential nucleotide conjugate-template complexes (e.g. Tm plus/minus 5° C.). Under such conditions, the creation and breakup of nucleotide conjugate-template complexes is in equilibrium. This allows for rapid interchange of nucleotide conjugates at a template. Binding within potential nucleotide conjugate-template complexes is reversible, and complexes are repeatedly formed and disaggregated, owing to the reaction conditions.

In a further embodiment, the reaction temperature preferably lies above the Tm of potential nucleotide conjugate-template complexes (e.g. Tm plus 5° C.). If relatively short B-segments (preferably from 3 to 15 base pairs) are employed, the bond between an oligonucleotide and another single-stranded nucleic acid chain is not particularly stable. This allows for rapid interchange of nucleotide conjugates at a nucleic acid chain to be labeled.

In a further embodiment of the invention, at least four compositions are employed, of which each composition comprises nuc-macromolecules with uniform nuc-component, at least one uniform marker, and different oligonucleotides. For example, four compositions of nuc-macromolecules are employed, of which a first composition has a dATP-nuc-component, a second composition has a dCTP-nuc-component, a third composition has a dGTP-nuc-component, a fourth composition has a dUTP-nuc-component.

In a further embodiment of the invention, at least one composition of such nucleotide conjugates is incubated with at least one primer-template complex and at least one polymerase under conditions that allow for reversible binding of the oligonucleotide components of the conjugates to the single-stranded portions of the primer-template complexes, as well as for incorporation of a complementary nucleoside triphosphate into the primer.

In a further embodiment of the invention, at least four compositions of nucleotide conjugates (e.g. dATP-population, dCTP-population, dGTP-population and dUTP-population) are incubated with at least one primer-template complex and at least one polymerase under conditions that allow for reversible binding of the oligonucleotide components of the nucleotide conjugates to a single-stranded portion of the primer-template complex, as well as for incorporation of a complementary nucleoside triphosphate into the primer. Each of these populations has at least one nucleoside triphosphate moiety, as well as an oligonucleotide population characteristic for this nucleoside triphosphate (FIG. 4-7)

Particular embodiments of the invention can be combined with one another so as to produce structures of nucleotide conjugates that contain advantageous combinations. For example, nucleotide conjugates with oligonucleotides can be provided that in parts contain self-complementary double-stranded regions as well as B-segments for binding to nucleic acid chains that are to be labeled.

In one embodiment, additional nucleotides can be employed. Natural dNTP (dATP, dCTP, dGTP, dTTP), or analogs thereof (e.g. ddNTP), or labeled nucleotides (e.g. dUTP-16-biotin) can be employed for example.

In a further embodiment, still other modified nuc-macromolecules are employed, as described in applications Cherkasov et al WO2011050938, Cherkasov et al WO2005044836, Cherkasov et al WO2006097320, Cherkasov et al WO2008043426.

In a preferred embodiment, nucleotide conjugates are used in concentrations in the following ranges: 10 pmol/l-1 nmol/l, 1 nmol/l-10 nmol/l, 10 nmol/l-100 nmol/l, 100 nmol/l-1 μmol/l, 1 μmol/l-10 μmol/l, 10 μmol/l-1 mmol/l. Ranges between 10 nmol/l and 10 μmol/l are preferred in particular. These concentrations may relate to the concentration of the nuc-component of the nucleotide conjugates.

The novel nucleotide conjugates may be used in methods for the enzymatic synthesis of nucleic acid chains. In particular, these nucleotide conjugates are preferably used in methods for the labeling and sequencing of nucleic acid chains. Exemplary implementations of methods for labeling or sequencing by synthesis are known to persons skilled in the art.

A method for sequencing nucleic acid chains comprises the following steps for example:
a) preparation of at least one population of extendable nucleic acid chain-primer complexes (NAC-primer complexes)
b) incubation of at least one type of nucleotide conjugate and at least one type of polymerase together with the NAC-primer complexes provided in step (a) under conditions that allow for incorporation of nucleotide conjugates with complementary nucleobases (nuc-components), where each type of nucleotide conjugate possesses a characteristic marker.
c) separation of unincorporated nucleotide conjugates from the NAC-primer complexes
d) detection of the signals of the nucleotide conjugates incorporated into NAC-primer complexes
e) cleaving-off of the linker component as well as of the marker component from the nucleotide conjugates incorporated into the NAC-primer complexes
f) washing of the NAC-primer complexes
   repetition of steps (b) through (f) if required,

In one embodiment, the nucleic acid chains to be sequenced can be attached to a solid phase in a random arrangement, and at least a part of the NAC-primer complexes can be individually optically addressed (sequencing-by-synthesis following the methods of Helicos Biosciences or Genovoxx GmbH).

In one embodiment, the nucleic acid chains to be sequenced can be attached to a solid phase in a random arrangement and form micro-colonies with identical sequences in each colony (sequencing by synthesis Solexa method of Illumina). The steps involved in such methods are known to a person skilled in the art.

### 1.3 Detailed Description of the Invention

### Terms and Definitions

1.3.1 Macromolecular compound—a molecule or complex of molecules or a nanocrystal or nanoparticle, which has a molecular weight between 2 kDa and 20 kDa, 2 kDa and 50 kDa, 2 kDa and 100 kDa, 100 kDa and 200 kDa, 200 kDa and 1000 kDa or 1 MDa and 100 MDa or 100 MDa and 100 Gda. Examples of macromolecular compounds are nucleic acids, e.g. oligonucleotides with a length of more than 10 nucleotides, polynucleotides, polypeptides, proteins or enzymes, quantum dots, polymers like PEG, Mowiol, dextran, polyacrylate, nanogold particles and complexes comprising several macromolecules.

1.3.2 Low-molecular compound—a molecule or a molecule complex, which has a mass smaller than 2000 Da (2 kDa), e.g. biotin, natural nucleotides, dATP, dUTP, many dyes, like Cy3, rhodamine, fluorescein and conventionally modified nucleotides, like biotin-16-dUTP.

1.3.3 A nuc-macromolecule (a nucleotide conjugate) within the meaning of this application is a chemical structure (a nucleotide analog or a nucleotide conjugate), which comprises one or more nuc-components, one or more linker components, and at least a marker component:

(Nuc-Linker)ₙ-Marker

wherein:
Nuc is a nuc-component
Linker is a linker component
Marker is a marker component
n is a positive integer from 1 to 100
Nuc is a nucleotide or a nucleoside monomer (a nuc component)
Linker has a composition which is not restricted as long as substrate properties of the nucleotides are not lost. Its length ranges between 5 and 100 chain atoms.
Marker is a marker component, which comprises at least one nucleic acid sequence with the length between 3 and 200 nucleobases (an oligonucleotide).
   n is a positive interger from 1 to 100, wherein (n) can be an average number.

Further examples for the synthesis and application of nuc-macromolecules are presented in the applications: Cherkasov et al WO2011050938, Cherkasov et al WO 2005044836, Cherkasov et al WO2006097320, Cherkasov et al WO 2008043426, Cherkasov et al DE 10356837, Cherkasov et al DE 102004009704.

### 1.3.3.1 Nuc-Component

Nuc-component is a substrate for nucleotide or nucleoside accepting enzyme. A nuc-component can represent a nucleotide as well as a nucleoside. In the following, nucleotides will be described as example for both classes of the substances. Nucleosides can be converted into a nucleotide form with corresponding enzymes or via chemical methods.

In one embodiment, the nuc-component is a nucleotide monomer or a nucleoside monomer, which is coupled to the linker component. In principle, all conventional nucleotide variants that are suitable as a substrate for nucleotide-accepting enzymes can serve as nuc-component of the nuc-macromolecule so that naturally occurring nucleotides as well as modified nucleotides (nucleotide analogs) can be considered for the nuc-component. Modified nucleotides comprise base-, sugar- or phosphate-modified nucleotide analogs. Many examples are known to the person skilled in the art (“Nucleoside Triphosphates and their Analogs”, Morteza Vaghefi, 2005, ISBN 1-57444-498-0; “Deoxynucleoside analogs in cancer therapy” Godefridus 3. Peters, 2006, ISBN 1-58829-327-0; “Chemistry of nucleosides and nucleotides” Leroy B. Townsend, 1991, ISBN 0-306-43646-9; “Advanced organic chemistry of nucleic acids”, 1994, Shabarova, ISBN 3-527-29021-4; “Nucleotide Analogs” Scheit, 1980, ISBN 0-471-04854-2; “Nucleoside and Nucleic Acid Chemistry”, Kisakũrek 2000, “Anti-HIV Nucleosides” Mitsuya, 1997, “Nucleoside Analogs in cancer therapy”, Cheson, 1997). further examples for modifications of the nucleotides will also be cited in the text. The nuc-component preferentially comprises a base part (base), a sugar part (sugar) and optionally a phosphate part (phosphate). Base, sugar and phosphate can be modified, i.e. the basic structure resembles the natural occurring nucleotides, but comprises e.g. additional chemical groups. Examples for combinations of different nucleotide components are known to the person skilled in the art. Such nuc-components can be used in a variety of enzymatic and chemical reactions (G. Wright et al. Pharmac. Ther. 1990, v. 47, p. 447-).

In a preferred embodiment, the nuc-component is a substrate for DNA polymerase. In a another preferred embodiment, the nuc-component is a substrate for RNA polymerase. Variations of the nucleotides, which allow for such substrate properties, can be used as nuc-components. For example, substrates for nucleotide accepting enzymes, which lack a part of a conventional nucleotide, e.g. acyclic nucleotide analoga, can be used as nuc-components, too.

### 1.3.3.1.1 Variations of the Phosphate

In one embodiment the nuc-component is a nucleoside. In another embodiment the nuc-component represents a nucleoside-monophosphate. In another embodiment the nuc-component represents a nucleoside-diphosphate. In another embodiment the nuc-component is a nucleoside-triphosphate. Still higher numbers of phosphate groups in a nucleotide (e.g. tetraphosphate, pentaphosphate etc.) can be used.

The said phosphate modifications can be located at the 5′-position of the sugar, like nucleoside-triphosphates, or also at other positions of the sugar part of the nucleotide, e.g. at the 3′-position.

Optionally, the phosphate part of the nucleotide can comprise modifications, in one embodiment such modifications comprising a linker, for example (D. Jameson et al. Methods in Enzymology 1997, v. 278, p. 363-, A. Draganescu et al. J. Biol. Chem. 2000v.275, p. 4555-). In another embodiment of the invention, the phosphate part of the nuc-component comprises thiotriphosphate derivates (Burges et al. PNAS 1978v. 75, p. 4798-).

In another embodiment of the invention, the phosphate part of the nuc-component comprises protected phosphate groups (e.g. phosphoroamidites).

In one embodiment, the phosphate part represents a linkage between the nuc-component and the linker component of the nuc-macromolecule.

### 1.3.3.1.2 Variations of the Base

The nuc-component can be natural nucleotide or nucleoside occurring in the nucleic acids in nature or their analogs, preferentially participating at the Watson-Crick base-pairing, e.g. adenine, guanine, thymine, cytosine, uracil, inosine or modified bases like 7-deazaadenine, 7-deazaguanine, 6-thioadenine (as referred above). Optionally, the base comprises modifications. In one embodiment, such modifications comprise for example a linker, e.g. amino-propargyl-linker or amino-allyl-linker. Further examples of linkers are known (Ward et al. U.S. Pat. No. 4,711,955, G. Wright et al. Pharmac. Ther. 1990, v. 47, p. 447-, Hobbs et al. U.S. Pat. No. 5,047,519 or other linkers e.g. Kievan U.S. Pat. No. 4,828,979, Seela U.S. Pat. No. 6,211,158, U.S. Pat. No. 4,804,748, EP 0286028, Hanna M. Method in Enzymology 1996 v.274, p. 403, Zhu et al. NAR 1994 v.22 p. 3418, Jameson et al. Method in Enzymology, 1997, v. 278, p. 363-, Held et al. Nucleic acid research, 2002, v. 30 p. 3857-, Held et al. Nucleosides, nucleotides & nucleic acids, 2003, v. 22, p. 391, Short U.S. Pat. No. 6,579,704, Odedra WO 0192284). In one embodiment, a linker coupled to the base represents a connection part between the nuc-component and the linker component of the nuc-macromolecule. Further modifications of the base are described for example in the catalogue of Trilink Biotechnologies, Inc. San Diego, USA, and are presented in “Nucleoside triphosphates and their analogs”, Morteza Vaghefi, 2005 ISBN 1-57444-498-0.

### 1.3.3.1.3 Variations of the Sugar

Different variations of the sugar part of the nucleotides, which are used e.g. in the diagnostics, therapy or research, are known to the person skilled in the art. Such variations comprise ribose, 2-deoxyribose or 2′,3′-dideoxyribose. Optionally, the sugar part comprises modifications (M. Metzker et al. Nucleic Acid Research 1994, v. 22, p. 4259-, Tsien WO 91/06678). In one embodiment, such modifications comprise for example a linker. The modifying group can be optionally be reversibly coupled to the sugar part (Hovinen et al. J. Chem. Soc. Prking Trans. 1994, s. 211-, Canard U.S. Pat. No. 5,798,210, Kwiatkowski U.S. Pat. No. 6,255,475, Kwiatkowski WO 01/25247, Ju et al. U.S. Pat. No. 6,664,079, Fahnestock et al. WO 91066678, Cheeseman U.S. Pat. No. 5,302,509, Parce et al. WO 0050642, Milton et al. WO 2004018493, Milton et al. 2004018497).

In one embodiment, the linker coupled to the sugar part represents the connection between the nuc-component and the linker component of the nuc-macromolecules.

In another embodiment, the sugar part comprises for example the following modifications: optionally the 3′-OH-Group or the 2′-OH-Group can be substituted by the following atoms or groups: halogen atoms, hydrogen atoms, amino- or mercapto- or azido groups (Beabealashvilli et al. Biochem Biophys Acta 1986, v. 868, p. 136-, Yuzhanov et al. FEBS Lett. 1992 v. 306, p. 185-).

In another embodiment, the nuc-component comprises acyclic nucleotide or nucleoside modifications (A. Holy Current Pharmaceutical Design 2003 v. 9, p. 2567-, G. Wright et al. Pharmac. Ther. 1990, v. 47, p. 447-). In another embodiment, the sugar part comprises a double bond.

In this application, the following abbreviations will be used for 2′-deoxynucleotides: dUTP for 2′-deoxyuridine-triphosphate, dCTP for 2′-deoxycytidine-triphosphate, dATP for 2′-deoxyadenosine-triphosphate, dGTP for 2′-deoxyguanosine-triphosphate.

Ability of nuc-component or its lack to be further extended by a polymerase is an important property of nucleotide conjugates. In one preferable embodiment of invention, nucleotide analoga are used as terminators of the enzymatic synthesis. An example for such analoga are ddNTP-Analoga, e.g. 2′,3′-dideoxy-UTP. A person skilled in the art should know other examples for terminators.

### 1.3.3.1.4 Linking of the Nuc-Component and Linker

The nuc-component is linked to the linker at a coupling position. In one embodiment, this coupling position of the linker on the nuc-component is located on the base. In another embodiment, the linker is attached to the sugar (e.g. ribose or deoxyribose). In another embodiment of the invention, the linker is attached to the terminal phosphate group of the phosphate-moiety of the nuc-component.

The linkage between the linker component and the nuc-component is preferentially covalent.

If the coupling position is on the base, then the following positions are preferable: position 4 or 5 for pyrimidine bases and positions 6,7,8 for purine bases. (Ward et al. U.S. Pat. No. 4,711,955, G. Wright et al. Pharmac. Ther. 1990, V. 47, S. 447-, Hobbs et al. U.S. Pat. No. 5,047,519 or other linker e.g. Kievan U.S. Pat. No. 4,828,979, Seela U.S. Pat. No. 6,211,158, U.S. Pat. No. 4,804,748, EP 0286028, Hanna M. Method in Enzymology 1996 v. 274, S.403, Zhu et al. NAR 1994 v. 22 S.3418, Jameson et al. Method in Enzymology, 1997, v. 278, S. 363-, Held et al. Nucleic acid research, 2002, v. 30 3857-, Held et al. Nucleosides, nucleotides & nucleic acids, 2003, v. 22, S. 391, Short U.S. Pat. No. 6,579,704, Odedra WO 0192284). Further examples for modifications on the base are represented in “Nucleoside triphosphates and their analogs”, Morteza Vaghefi, 2005 ISBN 1-57444-498-0; On sugar, positions 2′, 3′, 4′ or 5′ can serve as coupling positions. The coupling to the phosphate groups can proceed for example via alpha, beta, or gamma phosphate groups. Examples for coupling positions on the base are described in Short WO 9949082, Balasubramanian WO 03048387, Tcherkassov WO 02088382 (also see commercially available nucleotides e.g. from Amersham, Roche, Trilink Technologies, Jena Bioscience), on the ribose in Herrlein et al. Helvetica Chimica Acta, 1994, v. 77, p. 586, Jameson et al. Method in Enzymology, 1997, v. 278, p. 363, Canard U.S. Pat. No. 5,798,210, Kwiatkowski U.S. Pat. No. 6,255,475, Kwiatkowski WO 01/25247, Parce WO 0050642, on phosphate groups in Jameson et al. Method in Enzymology, 1997, v. 278, p. 363.

The location of the coupling position depends on the area of application of the nuc-macromolecules. For example, coupling positions on the sugar or on the base are preferable in cases where the marker is intended to stay coupled to the nucleic acid strand. The coupling to the gamma or beta phosphate groups can be used for example in cases where the marker has to be separated during the incorporation of the nuc-macromolecule.

The linking between the nuc-component and the linker component results for example via a coupling unit (L) that is a part of the linker component.

In one embodiment, the linkage between the nuc-component and the linker is stable, e.g. resistant to temperatures up to 130° C., pH-ranges from 1 to 14 and/or resistant to hydrolytical enzymes (e.g. proteases or esterases). In another embodiment of the invention, this linkage between the nuc-component and the linker component is cleavable under mild conditions.

This cleavable linkage allows removal of the linker components and the marker components. This can be advantageous for example for methods of sequencing by synthesis, like pyrosequencing, BASS (base addition sequencing schema) (Canard et al. U.S. Pat. No. 5,798,210, Rasolonjatovo Nucleosides & Nucleotides 1999, v. 18, p. 1021, Metzker et al. NAR 1994, v. 22, p. 4259, Welch et al. Nucleosides & Nucleotides 1999, v. 18, p. 19, Milton et al. WO 2004018493, Odedra at al. WO 0192284) or single molecule sequencing Tcherkassov WO 02088382. The choice of the cleavable linkage is not restricted insofar as it remains stable under conditions of enzymatic reaction, does not result in irreversible damage of the enzyme (e.g. polymerase) and is cleavable under mild conditions. “Mild conditions” is understood to mean conditions that do not result in damage of nucleic acid-primer complexes wherein, for example, the pH-range is preferentially between 3 and 11 and the temperature is between 0° C. and the temperature value (x). This temperature value (x) is dependent upon the Tm of the nucleic acid—primer complex (where Tm is the melting temperature) and is calculated for example as Tm (nucleic acid primer complex) minus 5° C. (e.g. Tm is 47° C., then the (x)-value is 42° C.; ester, thioester, acetales, phosphoester, disulfide linkages and photolabile compounds are suitable as cleavable linkages under these conditions).

Preferentially, the said cleavable linkage comprises chemical or enzymatic cleavable linkages or photolabile compounds. Ester, thioester, tartrate, disulfide, Diol- (z.B.—CH₂(OH)—CH₂(OH)—), and acetal linkages are preferred as examples of chemical cleavable groups (Short WO 9949082, “Chemistry of protein conjugation and crosslinking” Shan S. Wong 1993 CRC Press Inc., Herman et al. Method in Enzymology 1990v. 184 p. 584, Lomant et al. J. Mol. Biol. 1976 v. 104 243, “Chemistry of carboxylic acid and esters” S. Patai 1969 Interscience Publ., Pierce Catalog). Examples for photolabile compounds are described in Rothschild WO 9531429, “Protective groups in organic synthesis” 1991 John Wiley & Sons, Inc., V. Pillai Synthesis 1980 p. 1, V. Pillai Org. Photochem. 1987 v. 9 p. 225, Dissertation “Neue photolabile Schutzgruppen für die lichtgesteuerte Oligonucleotidsynthese” H. Giegrich, 1996, Konstanz, Dissertation “Neue photolabile Schutzgruppen fũr die lichtgesteuerte Oligonucleotidsynthese” S. M. Bühler, 1999, Konstanz).

### 1.3.3.1.5 Number of the Linked Nuc-Components

In one embodiment of the invention, only one nuc-component is coupled per nuc-macromolecule. In another embodiment of the invention, several nuc-components are coupled per nuc-macromolecule. If several nuc-components are coupled, they can be identical or different, whereas the average number of the nuc-components per nuc-macromolecule can range for example from 2 to 5, 5 to 10, 10 to 25, 25 to 50, 50 to 100.

### 1.3.3.2 Linker Component

The function of the linker is to link a nuc-component and a marker component in such a way that substrate properties of the nuc-component are retained for nucleotide accepting enzymes even after the coupling of a macromolecular marker.

The terms “linker” and “linker component” will be used synonymously in this application and comprise the whole structural part of the nuc-macromolecule between the nuc-component and the marker component. The exact composition of the linker is not limited and can vary. In one embodiment, the linker is preferentially hydrophilic.

### 1.3.3.2.1 Linker Length

An average linker length ranges between 2 to 5, 5 to 10, 10 to 20, 20 to 30, 30 to 40, 40 to 50, 50 to 60, 60 to 70, 70 to 80, 80 to 90, 90 to 100, 50 to 100, 100 to 1000 atoms (chain atoms), so that an average linker length amounts to between 2 to 5, 5 to 10, 10 to 20, 20 to 30, 30 to 40, 40 to 50, 50 to 60, 60 to 70, 70 to 80, 80 to 90, 90 to 100, 50 to 100, 100 to 1000 angstroms (measured on a molecule potentially stretched-out as much as possible).

If a nuc-macromolecule comprises several linker components, these linker components can be of the same or different lengths relative to each other.

Some parts of the linkers can comprise rigid areas and other parts can comprise flexible areas.

### 1.3.3.2.2 Short Linker

In a preferred embodiment, nuc-macromolecules have a short linker. Its length comprieses the ranges between 2 to 5, 5 to 10, 10 to 20, 20 to 30, 30 to 40, 40 to 50 chain atoms. Such linkers can carry functional groups, as for example amino, carboxy, mercapto, hydroxy groups, alkyn-, isothiocyanat-, aldehyd- or azid-group. Such group can be provided in reactive form such as NHS-ester for carboxy group. Further molecules can be coupled to these groups. In one embodiment, cross-linker are bound to the short linker so that resulting nuc-component can be further reacted with other substances such as macromolecular linker component or marker component. Examples of short linkers coupled to the nucleotides are known to the person skilled in the art (“Nucleoside triphosphates and their analogs”, Morteza Vaghefi, 2005 ISBN 1-57444-498-0, Ward et al. U.S. Pat. No. 4,711,955, G. Wright et al. Pharmac. Ther. 1990, V. 47, S. 447-, Hobbs et al. U.S. Pat. No. 5,047,519 or other linker e.g. Kievan U.S. Pat. No. 4,828,979, Seela U.S. Pat. No. 6,211,158, U.S. Pat. No. 4,804,748, EP 0286028, Hanna M. Method in Enzymology 1996 v. 274, S.403, Zhu et al. NAR 1994 v. 22 S.3418, Jameson et al. Method in Enzymology, 1997, v. 278, S. 363-, Held et al. Nucleic acid research, 2002, v. 30 3857-, Held et al. Nucleosides, nucleotides & nucleic acids, 2003, v. 22, S. 391, Short U.S. Pat. No. 6,579,704, Odedra WO 0192284). The linker can contain one or several units of polymers, as for example amino acids, sugars, PEG units or carboxylic acids. The coupling unit (L) of a long linker can serve as further examples of short linkers (see below). Examples for cross-linker are known to an expert (“Chemistry of protein conjugation and crosslinking” Shan S. Wong 1993). Many cross-linker are commercially available, e.g. from Invitrogen (Lifescience Technologies, Pierce Biotech, Iris-Biotech). Examples of coupling of different substances to macromolecules such as oligonucleotides are also known (Y. Singh et al Chem. Soc. Rev. 2010, 39, 2054-). It should be obvious to an expert that the linker between the nuc-component and the marker component can be assembled in several chemical steps.

Still further examples for short linkers between a nuc-component and a marker are represented by an example of linkage between a nucleoside triphosphate (NUK) and an oligonucleotide (OLN):
NUC-NH-OLN, NUC-O-OLN, NUC-S-OLN, NUC-SS-OLN, NUC-CO—NH-OLN, NUC-NH—CO-OLN, NUC-CO—O-OLN, NUC-O—CO-OLN, NUC-CO—S-OLN, NUC-S—CO-OLN, NUC-P(O)₂—OLN, NUC-Si—OLN, NUC-(CH₂)ₙ—OLN, NUC-(CH₂)ₙ—OLN, NUC-A-(CH₂)ₙ—OLN, NUC-(CH₂)ₙ—B-OLN, NUC-(CH═CH—)ₙ—OLN, NUC-(A-CH═CH—)ₙ—OLN, NUC-(CH═CH—B—)ₙ—OLN, NUC-A-CH═CH—(CH₂—)ₙ—OLN, NUC-(—CH═CH—CH₂)ₙ—B-OLN, NUC-(—CH═CH—CH₂—CH₂)ₙ—B-OLN, NUC-(—O—CH₂—CH₂)ₙ—B-OLN, NUC-A-(—O—CH₂—CH₂)ₙ—OLN, NUC-A-(—O—CH₂—CH₂)ₙ—B-OLN, NUC-(C≡C—)ₙ—OLN, NUC-(A-C≡C—)ₙ—OLN, NUC-(C≡C—B—)ₙ—OLN, NUC-A-C≡C—(CH₂—)ₙ—OLN, NUC-(—C≡C—CH₂)ₙ—B-OLN, NUC-(—C≡C—CH₂—CH₂)ₙ—B-OLN,
where NUC is the nuc-component; OLN is an oligonucleotide; A and B comprises the following structural elements: —NH—, —O—, —S—, —SS—, —CO—NH—, —NH—CO—, —CO—O—, —O—CO—, —CO—S—, —S—CO—, —P(O)₂—, —Si—, —(CH₂)ₙ—, a photolabile group; (n) is a number from 1 to 5

This examples are presented only for illustration purpose without intention to limit the structure of the linker.

### 1.3.3.2.3 Long Linker

In another preferred embodiment of the invention, a long linker having a length of more than 50 chain atoms is used. The linker component has has in its structure, for example, the following components:

1) coupling unit (L)
2) hydrophilic or water soluble polymer
3) coupling unit (T)

The subdivision of the linker in separate parts is purely functional and should serve merely for better understanding of the structure. Depending on the approach, particular structures can be considered as one functional part or as another.

The coupling unit (L) has the function of linking the linker component and the nuc-component. Short, non-branched compounds from 1 to 20 atoms in length are preferred. The particular structure of the coupling unit (L) depends on the coupling position of the linker to the nucleotide and on the particular polymer of the linker. Several examples of coupling units (L) are shown in examples 1 to 33 of this application. Many conventionally modified nucleotides comprise a short linker; these short linkers are further examples of coupling units (L), e.g. short linker on the base: Short WO 9949082, Balasubramanian WO 03048387, Tcherkassov WO 02088382 (see also commercially available nucleotides from e.g. Amersham or Roche), short linker on the ribose as described in Herrlein et al. Helvetica Chimica Acta, 1994, v. 77, p. 586, Jameson et al. Method in Enzymology, 1997, v. 278, p. 363, Canard U.S. Pat. No. 5,798,210, Kwiatkowski U.S. Pat. No. 6,255,475, Kwiatkowski WO 01/25247, Ju et al. U.S. Pat. No. 6,664,079, Parce WO 0050642, and short linker on phosphate groups as described in Jameson et al. Method in Enzymology, 1997, v. 278, p. 363.

Still further examples for the coupling unit (L) are presented in the following:
R₆—NH—R₇, R₆—O—R₇, R₆—S—R₇, R₆—SS—R₇, R₆—CO—NH—R₇, R₆—NH—CO—R₇, R₆—CO—O—R₇, R₆—O—CO—R₇, R₆—CO—S—R₇, R₆—S—CO—R₇, R₆—P(O)₂—R₇, R₆—Si—R₇, R₆—(CH₂)ₙ—R₇, R₆—(CH₂)ₙ—R₇, R₆-A-(CH₂)ₙ—R₇, R₆—(CH₂)ₙ—B—R₇, R₆—(CH═CH—)ₙ—R₇, R₆-(A-CH═CH—)ₙ—R₇, R₆—(CH═CH—B—)ₙ—R₇, R₆-A-CH═CH—(CH₂—)ₙ—R₇, R₆—(—CH═CH—CH₂)ₙ—B—R₇, R₆—(—CH═CH—CH₂—CH₂)ₙ—B—R₇, R₆—(C≡C—)ₙ—R₇, R₆-(A-C≡C—)ₙ—R₇, R₆—(C≡C—B—)ₙ—R₇, R₆-A-C≡C—(CH₂—)ₙ—R₇, R₆—(—C≡C—CH₂)ₙ—B—R₇, R₆—(—C≡C—CH₂—CH₂)ₙ—B—R₇,
where R₆ is the nuc-component; R₇ is a polymer; A and B comprises the following structural elements: —NH—, —O—, —S—, —SS—, —CO—NH—, —NH—CO—, —O—CO—, —CO—S—, —S—CO—, —P(O)₂—, —Si—, —(CH₂)ₙ—, a photolabile group; (n) is a number from 1 to 5

The coupling unit L is covalently linked to the nuc-component on the one side. On its other side further parts of the linker, for example, a hydrophilic polymer or directly the coupling unit (T) or directly the marker can be bound.

In the following, the coupling of the polymer, as a part of the linker is explained as example. The character of the linkage with the polymer depends on the kind of polymer. In a preferred embodiment, the ends of the polymer comprises reactive groups, for example NH2 (amino), OH (hydroxy), SH (mercapto), COOH (carboxy), CHO (aldehyde), acrylic, maleimide, or halogen groups, or alkyn-, Isothiocyanat- or Azid-Group. Such groups can be provided as a reactive form, e.g. NHS-ester for carboxy-group. Such polymers are commercially available (e.g. Fluka, Iris-Biotech, Nanocs inc, Pierce Biotech). Some examples for the coupling of polymers to the coupling unit are shown in the examples.

In a preferred embodiment, the water-soluble polymer represents the major part of the linker component. It is a polymer, preferentially hydrophilic, consisting of the same or different monomers.

Examples of suitable polymers are polyethylene-glycol (PEG), polyamides (e.g. polypeptides), polysaccharides and their derivates, dextran and its derivates, polyphosphates, polyacetates, poly(alkyleneglycols), copolymers with ethylenglycol and propyleneglycol, poly(olefinic alcohols), poly(vinylpyrrolidones), poly(hydroxyalkylmethacrylamides), poly(hydroxyalkylmethacrylates), poly(x-hydroxy acids), polyacrylic acid and their derivates, poly-acrylamide and its derivates, poly(vinylalcohol), polylactic acid, polyglycolic acid, poly(epsilon-caprolactones), poly(beta-hydroxybutyrates), poly(beta-hydroxyvalerate), polydioxanones, poly(ethylene terephthalates), poly(malic acid), poly(tartronic acid), poly(ortho esters), polyanhydrides, polycyanoacrylates, poly(phosphoesters), polyphosphazenes, hyaluronidate, and polysulfones.

In one embodiment, the polymer-part comprises branched polymers. In an other embodiment, the polymer-part comprises non-branched or linear polymers. The polymer can consist of several parts of different length, each part consisting of the same monomers with the monomers in different parts being different. To a person skilled in the art, it should seem obvious that for a macromolecular linker, it is often possible to determine only an average mass, so that the data regarding the mole masses represent an average (“Makromoleküle, Chemische Struktur and Synthesen”, Volume 1, 4, H. Elias, 1999, ISBN 3-527-29872-X). For this reason, often there is no exact mass information for nuc-macromolecules.

### 1.3.3.2.4 Linker Coupling in a Nuc-Macromolecule

The linker is connected to the nuc-component on one side and to the marker component on the other side. The linker can have coupling units at his ends which fulfill this connecting function. The connection to the nuc-component was discussed above. The connection between the linker and the marker components is provided by coupling unit T. Short, non-branched connections no more than 20 atoms in the length are preferred. The respective structure of the coupling unit T depends upon the coupling position on the marker component and upon the respective polymer of the linker.

The coupling unit T is covalently connected to the polymer. The kind of the coupling depends on the kind of the polymer. In a preferred embodiment, the polymer has reactive groups at its ends such as NH2 (amino), OH (hydroxy), SH (mercapto), COOH (carboxy), CHO (aldehyde), acrylic, maleimide, or halogen groups, or alkyn-, Isothiocyanat- or Azid-Groups. Such groups can be provided as a reactive form, e.g. NHS-ester for carboxy-group. Such polymers are commercially available (e.g. Fluka, Iris-Biotech, Nanocs inc, Pierce Biotech). Some examples of the coupling units L are shown in Cherkasov et al WO 2005044836, Cherkasov et al WO2006097320, Cherkasov et al WO 2008043426, Cherkasov et al DE 10356837, Cherkasov et al DE 102004009704. For further examples of the chemical and affine connections please refer to the literature: “Nucleoside triphosphates and their analogs”, Morteza Vaghefi, 2005 ISBN 1-57444-498-0; “Chemistry of protein conjugation and crosslinking” Shan S. Wong in 1993, “Bioconjugation: protein coupling techniques for the biomedical sciences”, M. Aslam, in 1996.

The linker can also comprise other functional groups or parts, for example one or several groups that are cleavable under mild conditions, see also Cherkasov et at WO 2005044836, Cherkasov et at WO2006097320, Cherkasov et at WO 2008043426, Cherkasov et al DE 10356837, Cherkasov et at DE 102004009704.

A cleavable group within the linker allows the removal of a part of the linker and the marker component. After a cleavage reaction, a linker residue remains coupled to the nuc-component. Examples of cleavable groups are shown in Section 1.3.3.1.4.

### 1.3.3.3 Marker Component

Examples of signal-generating marker components and for the composition of the marker of nuc-macromolecules are provided in applications Cherkasov et at WO 2005044836, Cherkasov et at WO2006097320, Cherkasov et at WO 2008043426.

### Oligonucleotide Component of the Nucleotide Conjugate

In one embodiment, a marker component contains at least one oligonucleotide (FIG. 1). This oligonucleotide may contain DNA, PTO, RNA, PNA, LNA or comprise other modifications of nucleic acid chains with the capacity for base pairing.

In one embodiment, the oligonucleotide is partially or completely double-stranded.

This may be achieved, for example, by means of self-complementary regions within the oligonucleotide, or by means of hybridization with another predominantly or fully complementary oligonucleotide.

Such double-stranded oligonucleotide structures can prevent a polymerase from incorporating two and more nucleotide conjugates in adjacent positions. Synthesis is arrested owing to the steric effect of the oligonucleotide. Many examples of arresting synthesis during replication by means of hairpin-structures within the matrices are known to a person skilled in the art. One surprising result of this invention is that such hairpin-structures and fully double-stranded oligonucleotides within a nucleotide conjugate can likewise arrest synthesis.

By cleavage of the linker of an incorporated nucleotide conjugate this steric hindrance is removed, with the result that polymerase can proceed with synthesis.

In one embodiment of the invention, the oligonucleotide comprises at least one single-stranded sequence segment that can undergo complementary binding to single-stranded nucleic acid chains. This binding preferably takes place via hybridization to the nucleic acid chain to be labeled. The length of this segment of the oligonucleotide must be adjusted to the respective assay conditions. This length includes the following ranges (counted in the nucleobases): 2-4, 4-6, 6-8, 8-10, 10 to 15, 15 to 20, 20 to 50, 50 to 100.

In a further embodiment, an oligonucleotide comprises more than one such sequence segment.

The nucleobase composition of this segment is preferably chosen in such a manner that the discrimination ability of such a sequence segment of the oligonucleotide is kept deliberately low under prevailing reaction conditions.

This can be achieved, for example, with short stretches of 4 to 8 DNA nucleotide monomers at room temperature conditions. A person skilled in the art knows similar examples where low discrimination is achieved, for example with hexamer primers.

In another embodiment, the oligonucleotide structure contains one or several homopolymer segments (e.g. 5 to 50 adenosine-nucleobases, or 5 to 50 cytosine nucleobases, or 5 guanosine nucleobases, or 5 to 50 thymidine-nucleobases). Homopolymer segments of this type can undergo relatively non-specific base pairing with other homopolymer-containing sequence regions. In another embodiment, the oligonucleotide structure includes one or several short repetitive sequence segments, for example 2 to 100 segments with a repetitive sequence, such as AATCC. Such repeats can likewise undergo relatively non-specific binding with correspondingly complementary nucleic acid segments.

The specificity of binding to the respective nucleic acid chain may vary depending on the nature of the oligonucleotide moiety (i.e., DNA, PTO, RNA, PNA or LNA). A person skilled in the art knows that PNA-based oligonucleotides exhibit stronger affinity for complementary regions than DNA-based oligonucleotides of the same length and composition for example.

In one embodiment, coupling of the oligonucleotide within the nuc-macromolecule is effected at one of the two ends of the oligonucleotide (FIG. 11), for example at the 5′-end or at the 3′-end. Examples of coupling an oligonucleotide at one of its ends are known to a person skilled in the art. In another embodiment of the invention, the coupling to other parts of the nuc-macromolecule (e.g. of the nuc-component) is effected within the interior region of the oligonucleotide.

The respective linker between the nuc-component and a oligonucleotide may for example be attached at one of the bases of the oligonucleotide or at one of its backbone monomers (e.g. at the sugar or the phosphate in case of an DNA backbone, or at an amino acid in case of a PNA backbone, or at the sulfur atom of a PTO-backbone). A person skilled in the art knows different ways in which moieties may be coupled to an oligonucleotide at various positions.

The oligonucleotide segment that can undergo non-specific binding with nucleic acid chains can be flanked by other sequence segments at its 5′- or its 3′-end. These flanking regions may consist of the same monomers as the binding segment or their composition may differ from the binding segment (e.g. DNA, PTO, PNA, LNA, RNA). These flanking sequence segments may be 1 to 5, 5 to 10, 10 to 15, 15 to 20, 20 to 30, 30 to 100, or more than 100 nucleobases in length. They may serve as a spacer or they may perform functions of the marker component for example (see Cherkasov et al WO 2005044836, Cherkasov et al WO2006097320, Cherkasov et al WO 2008043426).

A linker that connects the nuc-component with the oligonucleotide can be attached to such a flanking region for example.

The oligonucleotide may in parts contain self-complementary regions, such as hairpin structures or loops (FIG. 8). In a preferred embodiment of the invention, the oligonucleotide participates in the formation of a structure of the type of the so-called “molecular beacon” (on the properties of molecular beacons: Bonnet et al. PNAS 1999, v96. 6171-). The self-complementary regions of such molecular beacons are typically between 4 to 6, 6 to 8, 8 to 10, 10 to 15, 15 to 30 nucleotides in length. Several self-complementary regions can be present in one oligonucleotide, for example from 1 to 10. The sequence compositions of these self-complementary regions may be different.

A person skilled in the art knows oligonucleotide modifications that influence binding to nucleic acid chains. Such modifications include, for example, “minor groove binders”.

In one embodiment, the 3′-OH position of the oligonucleotide (or of the flanking oligonucleotide) has been blocked by means of a chemical group. A person skilled in the art knows many examples of modifications of the 3′-OH group of oligonucleotides. They include, for example, the following moieties: 2′,3′-dideoxy-ribose, a phosphate group, a biotin residue, an amino linker, a fluorescent dye, a peptide chain, a quencher. Various modifications instead of a 3′-OH group can be incorporated into an oligonucleotide, such as an amino group, a halogen atom, an azide group, etc. The oligonucleotide in this embodiment cannot be extended by a polymerase; it therefore it has no primer function.

In another embodiment, the 3′-OH end of the oligonucleotide has not been blocked and extension by a polymerase can take place.

Preferably, an oligonucleotide of nuc-macromolecules contains nucleic acid chains with a total length within the following ranges: from 3 to 6, 6 to 9, 9 to 12, 12 to 14, 14 to 16, 16 to 18, 18 to 20, 20 to 25, 25-30, 30-40, 40-50, 50-60, 60-70, 70-100, 100 to 200 nucleobases.

In a preferred embodiment of the invention, the sequences of the oligonucleotides are chosen such that they are not able to bind to other types of nuc-macromolecules under the reaction conditions employed. Every type of nuc-macromolecule thus possesses its own oligonucleotide sequence, which is not complementary to other oligonucleotides.

The oligonucleotide may carry additional modifications, such as signal-generating or signal-transmitting molecules, like dyes, fluorescent dyes, biotin, or macromolecular compounds such as enzymes or nanocrystals for example.

Regarding the chemical synthesis of oligonucleotides and modifications thereof, a person skilled in the art may be referred to the following sources:

Singh et al Chem Soc Rev, 2010, v. 39, 2054-, “Oligonucleotide synthesis, methods and applications” Piet Herdewijn, 2004, ISBN 1-58829-233-9, “Protocols for oligonucleotide conjugates, synthesis and analytical techniques” Sudhir Agrawal, 1993, ISBN 0-89603-252-3, “Protocols for oligonucleotide conjugates, synthesis and properties” Sudhir Agrawal, 1993, ISBN 0-89603-247-7, “The aptamer handbook” Sven Klussmann, 2006, ISBN 10: 3-527-31059-2, “Pharmaceutical aspects of oligonucleotides” Patrick Couvreur, 2000, ISBN 0-748-40841-X,
“Triple Helix forming Oligonucleotides” Claude Malvy, 1999, ISBN 0-7923-8418-0, “Artificial DNA, methods and applications” Yury E. Khudyakov, ISBN 0-8493-1426-7
Probes (e.g. Oligonucleotides) Complementary to the Oligonucleotide Sequence of the Nuc-Macromolecule (FIG. 9).

In one embodiment of the invention, nucleotide conjugate structures include further nucleic acid chains that contain stretches with sequence-specific complementarity to the oligonucleotide moiety of the nuc-macromolecule (FIG. 8-10). These nucleic acid chains may be described as complementary oligonucleotides.

### Structure of Complementary Oligonucleotides

In one embodiment, the complementary oligonucleotide is composed of nucleobases. Nucleobases like adenine, cytosine, guanine, thymine, uracil (abbreviated as A, C, G, T, U), or analogs thereof linked to a sugar-phosphate backbone of the DNA or RNA type, or analogues thereof, such as PTO, PNA, LNA, can partake in sequence-specific binding to the nucleic acid strands.

If several complementary oligonucleotides are present within one type of nuc-macromolecule, individual sequence segments may be composed of different types of monomers, such that one antagonist oligonucleotide consists of DNA, another of PNA, etc.

To simplify the description, complementary oligonucleotides of the DNA type are discussed in detail. Other types of nucleic acid chains may be synthesized and employed according to rules for DNA oligonucleotides, which are known to a person skilled in the art.

The length of the complementary oligonucleotide preferably falls in the following ranges: 15 to 25, 25 to 50, 50 to 100, more than 100 base pairs.

The complementary oligonucleotide can be flanked by further sequence segments at the 5′-end or at the 3′-end that do not bind to the oligonucleotide of nucleotide conjugate. These flanking sequence segments may be from 1 to 5, 5 to 10, 10 to 15, to 20, from 20 to 30, or more than 30 nucleobases in length. They can serve as spacers or markers.

A person skilled in the art will be aware of further nucleotide modifications which influence the binding interaction of complementary nucleic acid chains. Such modifications include “minor groove binders” for example.

In one embodiment, the 3′-OH end of the complementary oligonucleotide (or of the flanking oligonucleotide) has been blocked by means of a chemical group. A person skilled in the art knows many examples of modifications of the 3′-OH group of oligonucleotides. They include, for example, the following moieties: 2′,3′-dideoxy-ribose, a phosphate group, a biotin residue, an amino linker, a fluorescent dye, a peptide chain, a quencher. Various modifications instead of an 3′-OH group can be incorporated into an oligonucleotide, such as an amino group, a halogen atom, an azide group, etc. The oligonucleotide in this embodiment cannot be extended by a polymerase; it therefore it has no primer function.

The complementary oligonucleotides may in parts contain self-complementary segments, such as hairpin structures or loops (FIG. 8). In a preferred embodiment of the invention, the antagonist-oligonucleotide participates in the formation of a structure of the type of the so-called “molecular beacon” (on the properties of molecular beacons: Bonnet et al. PNAS 1999, v96. 6171-).

In one embodiment, the binding of complementary oligonucleotides to nucleotide conjugates is effected prior to the incorporation reaction. In a further embodiment, the binding of complementary oligonucleotides to nucleotide conjugates only takes place after the incorporation reaction.

### 1.3.3.3.3 Signal Domain (Functions and Composition)

### Function of a Signal Domain

In one embodiment, the signal domain can have a signaling function. In another embodiment, it has a signal-transmitting function. In another embodiment, it has a catalytic function. In a further embodiment, the signal domain has more than one function and combines for example both signaling and signal-transmitting functions. Other combinations are obvious.

The signal domain having signaling function comprises constituents which have been assembled within a nuc-macromolecule during the chemical synthesis of a nuc-macromolecule: for examples see the applications Cherkasov et al WO 2005044836, Cherkasov et al WO2006097320, Cherkasov et at WO 2008043426, Cherkasov et at DE 10356837, Cherkasov et at DE 102004009704.

In one embodiment, the oligonucleotide of the nucleotide conjugate has a signal function: it may, for example, contain one or more fluorescent dyes.

In a further embodiment, the oligonucleotide of the nucleotide conjugate has a signal-transmitting function: it may contain at least one biotin residue for example, or it contains a sequence segment which can bind additional labeled oligonucleotides.

### 1.3.3.3.4 Core Component of the Marker

The core component has the function of connecting several structural elements of the nuc-macromolecules. For instance, the core component connects several marker units together or individual domains can be coupled throught the core component. In a further embodiment, linker components can be bound to the core component (FIG. 5). The term “core-component” is functional and serves for illustration of possible structures of nuc-macromolecules. Different chemical structures that connect linker and marker-units can be called core-component. Examples for constituents of the core component will be presented.

Further examples for the core component are presented in applications Cherkasov et al WO 2005044836, Cherkasov et al WO2006097320, Cherkasov et al WO 2008043426.

### 1.3.3.3.6 Coupling Between Linker and Marker

The connection between the linker component and the marker depends on the respective structures of the marker units or the structure of the core component. In one embodiment, the linker component is bound directly to the signal-giving or signal-transmitting marker unit. The marker can consist of only one or several marker units. In a further embodiment, one or several linker components are bound to the core component of the marker.

The connection between the linker component and the marker can be covalent as well as affine. Many examples are known to the specialist, e.g. “Bioconjugation: protein coupling techniques for the biomedical sciences”, M. Aslam, in 1996, ISBN 0-333-58375-2. “Chemistry of protein conjugation and crosslinking” Shan S. Wong in 1993 CRC Press Inc).

Covalent coupling: In one embodiment, the connection between the linker component and the marker can be resistant to, e.g., temperatures up to 130° C., pH ranges between 1 and 14, and/or resistant to hydrolytic enzymes (e.g. proteases, estarases). In another embodiment, the connection is cleavable under mild conditions.

According to some embodiments of this invention, macromolecular compounds used for the labeling of nucleotides comprise water-soluble polymers (see above). The linker of the nuc-macromolecules comprises water-soluble polymers too. A person skilled in the art should recognize that assignment of individual polymers to the linker or to the marker has a descriptive character.

### 1.3.3.3.7 Ratio of Nuc-Components in a Nuc-Macromolecule

One nuc-macromolecule can comprise on average 1 to 2, 2 to 5, 5 to 10, 10 to 30, 30 to 100 nuc-components.

In one embodiment, all nuc-macromolecules have the same number of nuc-components per one nuc-macromolecule. For instance, a maximum of 4 biotin molecules can be bound per one strepavidin molecule; at a saturating concentration of nuc-linker components, a uniform population of nuc-macromolecules can be obtained.

In another embodiment, a nuc-macromolecule population has a defined average number of nuc-components per one nuc-macromolecule, however, in the population itself there is dispersion in the actual occupation of the nuc-macromolecules by nuc-components. In this case, the number of nuc-components per one nuc-macromolecule displays an average.

### 1.3.3.3.8 Ratio of Marker Units in a Nuc-Macromolecule

The number of marker units in one nuc-macromolecule falls within the following ranges: 1 and 2, 2 and 5, 5 and 20, 20 and 50, 50 and 100, 100 and 500, 500 and 1000, 1000 and 10000, 10000 and 100000, or more than 100000.

In one embodiment, nuc-macromolecules have a definite number of signal-giving units per one marker. In another embodiment, a population of nuc-macromolecules has a varying number of marker units per one nuc-macromolecule and it does not need to have a definite value for every single nuc-macromolecule in a population. Further examples can be found in applications Cherkasov et at WO2011050938, Cherkasov et at WO2005044836, Cherkasov et at WO2006097320, Cherkasov et at WO2008043426.

### 1.3.6. Polymerases

In one embodiment, the nuc-macromolecules can be used as substrates for enzymes. Polymerases represent frequently used enzymes, which utilize nucleotides as substrates. They will be dealt with further as representative examples of other nucleotide-utilizing enzymes. One of the central abilities of polymerases consists in covalent coupling of nucleotide monomers to a polymer. Furthermore, the synthesis can be template-dependent (as for example DNA or RNA synthesis with DNA- or RNA-dependent polymerases) as well as independent of templates, e.g. terminal transferases Sambrook “Molecular Cloning” 3. Ed. CSHL Press in 2001).

If RNA is used as a substrate (e.g., mRNA) in the sequencing reaction, commercially available RNA-dependent DNA polymerases can be used, e.g. AMV reverse transcriptase (Sigma), M-MLV reverse transcriptase (Sigma), HIV reverse transcriptase without RNAse activity. For Klenow Fragment DNA polymerase a function as reverse transcriptase is also described. For certain applications, reverse transcriptases can be essentially free of RNAse activity (“Molecular cloning” in 1989, Ed. Maniatis, Cold Spring Harbor Laboratory), e.g. for use in mRNA labeling for hybridisation applications.

If DNA is used as a substrate (e.g. cDNA), all the following polymerases are suitable in principle: DNA-dependent DNA polymerases with or without 3′-5′ exonuclease activity (“DNA-Replication” in 1992 Ed. A. Kornberg, Freeman and company NY), e.g. modified T7-Polymerase for example of the type “Sequenase version 2” (Amersham Pharmacia Biotech), Klenow fragment of the DNA-Polymerase I with or without 3′-5′ exonuclease activity (New England Biolabs), T4 DNA Polymerase, phi29 DNA Polymerase, polymerase Beta of different origin (“Animal Cell DNA polymerases” in 1983, Fry M., CRC Press Inc, commercially available from Chimerx), thermostable polymerases such as, for example, Taq-Polymerase (New England Biolabs), Vent Polymerase, Vent exo minus Polymerase, Deep Vent Polymerase, Deep Vent exo minus Polymerase, Pfu Polymerase, Tli Polymerase, Tfl Polymerase, Tth Polymerase, Thermosequenase, Pwo-Polymerase, Terminator, Terminator I, Terminator II, Terminator III, Bst DNA Polymerase, Bst DNA Polymerase, Large Fragment, Phusion® High-Fidelity DNA Polymerase, Phusion® High-Fidelity Hot Start DNA Polymerase, Phire® Hot Start DNA Polymerase, Phire®Hot Start II DNA Polymerase, Phusion® Flash High-Fidelity DNA Polymerase, Crimson Taq DNA Polymerase, DyNAzyme™ EXT DNA Polymerase, DyNAzyme™ II Hot Start DNA Polymerase, 9° Nₘ DNA Polymerase etc. (for example from New England Biolabs, or from Promega, or from Roche, or from Qiagen).

Using modern genetic engineering methods, it is possible to construct polymerases which differ in their capabilities from naturally occurring enzymes, for example by the absence of certain activities or improved enzymatic parameters such as precision or processivity. An increasing number of companies manufacture such thermolabile and thermostable polymerases, which are used as optimized enzymes for PCR or other amplification or labeling methods. The basic functions of polymerases are retained, however: they are able to incorporate nucleotides into complementary strands during the synthesis. Such polymerases can also be used for the methods described. An expert is aware of how to bring about an optimization of the reaction conditions.

In one embodiment of the application, polymerases without 5′−3′exonuclease activity such as Klenow fragment exo minus, Vent exo minus, Bst-polymerase large fragment are preferentially used.

In one embodiment of the application, polymerases without 3′-5′ exonuclease activity such as Klenow fragment exo minus are preferentially used.

### 1.3.7 Cleavable Compound

A compound which is cleavable under mild conditions. This compound can represent a part in the linker and can be cleavable in one or several positions. It can be a chemically cleavable bond, such as, for example, disulfide, acetal, oxidative cleavable bonds (e.g. Linker comprising tartrate bond), thioester bonds (Short WO 9949082, Tcherkassov WO 02088382). It can also be a photo-chemically cleavable compound (Rothschild WO 9531429). It can also be an enzymatically cleavable compound (for instance, a peptide or polypeptide bond, Odedra WO 0192284), cleavable by peptidases, a poly- or oligo-saccharide bond, cleavable by disaccharidases, whereas the cleavage can be achieved by a specific enzyme between certain monomers of the cleavable bonds.

Several examples of cleavable compounds are known. The synthesis of such a compound is described, for instance, in (Tcherkassov WO 02088382, Metzker et al. Nucleic Acid Research 1994, v. 22, p. 4259-, Canard et al. Genes, 1994, v. 148, p. 1, Kwiatkowski U.S. Pat. No. 6,255,475, Kwiatkowski WO 0125247, Parce WO 0050642). A cleavable compound can be a part of the linker or can form the connecting part of the linker to the nucleotide, or the connecting part of the linker component to the marker component, or the connection between marker units and the core component.

1.3.8 DNA Deoxyribonucleic acid of different origin and different length (e.g. oligonucleotides, polynucleotides, plasmides, genomic DNA, cDNA, ssDNA, dsDNA)
1.3.9 RNA—Ribonucleic acid

### 1.3.10 PNA—Peptide Nucleic Acid

1.3.11 LNA—locked nucleic acids

### 1.3.12 Nucleotides

Nucleotides serve as substrates for polymerases in a template dependent synthesis reaction. They can be incorporated into a complementary strand.
dNTP—2′-deoxynucleoside triphosphate or their analoga, as a substrate for DNA polymerases and reverse-transcriptases, e.g. dATP, dGTP, dUTP, dTTP, dCTP, dITP or their analoga like 7-Deaza-dATP or 7-Deaza-dGTP. Also other analoga of naturally occurring 2′-deoxi-nucleoside-triphosphates can be used as substrates by DNA-polymerases.
NTP—Ribonucleoside triphosphate or their analoga, as a substrate for RNA polymerases, UTP, CTP, ATP, GTP.
Abbreviation “NT” is used for the description of the length of a particular nucleic acid sequence, e.g. 1000 NT. In this case “NT” means nucleoside monophosphates.

The plural is formed by the addition of the suffix “-s”; “NT” means, for example, “one nucleotide”, “NTs” means “several nucleotides”.

1.3.13 NAC—Nucleic acid chain. DNA or RNA, PNA, LNA

### 1.3.14 Term “the Whole Sequence”

The whole sequence is the sum of all the sequences to be analyzed in one experiment; it can comprise originally one or several NACs. Also, the whole sequence can display parts or equivalents of another sequence or sequence populations (e.g., mRNA, cDNA, Plasmid DNA with insert, BAC, YAC) and can originate from one species or various species. The “whole sequence” can comprise one or several target sequences.

### 1.3.15 NACF

The nucleic acid chains fragment (NSKF abbreviation stands for German “Nukleinsaurekettenfragment”) (DNA or RNA) which corresponds to a part of the whole sequence, NACFs—the plural form—nucleic acid chain fragments. The sum of the NACFs forms an equivalent to the whole sequence. The NACFs can be, for instance, fragments of the whole sequence (DNA or RNA), which result after a fragmentation step.

### 1.3.16 Primer Binding Site (PBS)

A PBS is the part of the target sequence to which the primer binds.

### 1.3.17 Reference Sequence

A reference sequence is an already known sequence, divergences from which in the analysed sequence or sequences (e.g. whole sequence) have to be determined. Reference sequences can be found in databases, such as, for example, the NCBI database.

### 1.3.18 Tm Melting Temperature

### 1.4. Important Aspects of the Invention are Presented Below

Aspect 1: nucleotide conjugates that comprise the following components: at least one nucleotide component (nuc-component), at least one oligonucleotide, and at least one linker between the nucleotide component and the oligonucleotide.

In one embodiment, the nucleotide component is attached to one of the ends of the oligonucleotide via a linker. In a further embodiment, the nucleotide component is attached at an internal position of the oligonucleotide via a linker. In one embodiment, the linker may be coupled to the base of the nucleotide component. In a further embodiment, the linker may be coupled to the sugar moiety of the nucleotide component.

Aspect 2: nucleotide conjugates in accordance with the aspect 1, wherein the linker employed is cleavable. For example, the linker contains a disulfide bond or a photolabile bond.

Aspect 3: nucleotide conjugates in accordance with the aspect 1, wherein the oligonucleotide contains self-complementary sequence segments. These sequence segments can span 4 to 10, 10 to 20, 20 to 40, or more than 40 bases. Preferably they are between 4 and 15 bases in length.

Aspect 4: nucleotide conjugates in accordance with the aspect 1, wherein at least one further complementary oligonucleotide is bound to the oligonucleotide. In one embodiment, the binding between the two oligonucleotides is achieved by hybridization of complementary regions of the oligonucleotides.

Aspect 5: nucleotide conjugates in accordance with any of the aspects 1 to 4, wherein at least one of the oligonucleotides is specifically labeled. The label may be a fluorescent dye for example.

In one embodiment of the invention, the 3′-end of said oligonucleotide is blocked by means of a chemical group, such as phosphate group or a dye.

Aspect 6: A reaction mixture or a composition for enzymatic synthesis of nucleic acid chains that comprises at least one of the nucleotide conjugates in accordance with the above aspects of the invention.

Aspect 6: A reaction mixture or a composition for enzymatic synthesis of nucleic acid chains that comprises at least four types of nucleotide conjugates in accordance with the above aspects of the invention, wherein the nuc-components of the nucleotide conjugates of the composition are selected from the following group of bases or their analogues: adenine, guanine, cytidine, uridine; and each type of nucleotide conjugate includes a particular characteristic marker.

Aspect 7: A reaction mixture or a composition for enzymatic synthesis of nucleic acid chains that comprises at least four populations of nucleotide conjugates in accordance with the above aspects of the invention, wherein each population of nucleotide conjugates is characterized by a uniform nucleobase for its nucleotide component, or analogues thereof (e.g. adenine, guanine, cytidine, uridine).

In one embodiment of the invention, a population of nucleotide conjugates with a single type of nucleobase for its nuc-component comprises multiple oligonucleotides. The number of oligonucleotides in a population of nucleotide conjugates spans the following ranges: 4 to 50, 50 to 500, 500 to 5000, 5000 to 10000, 10000 to 1000000, more than 1000000. It preferably falls into the range between 4 and 5000.

Nucleotide conjugates belonging to a population preferably possess at least one marker that is characteristic for this population. In one embodiment, this marker contains a fluorescent dye. In a further embodiment, the marker comprises a particular single-stranded sequence segment in the oligonucleotides.

In a further embodiment of the invention, the oligonucleotides of a population contain at least one variable sequence segment. This variable sequence segment differs among the oligonucleotides of the population. The number of variants of this segment depends on the length of the segment. The longer the sequence segment, the greater may be the degree of variability in this segment. In one embodiment, the multitude of variable sequence segments of the oligonucleotides of a population includes all possible nucleobase sequence permutations for this segment (randomized sequences). The number of possible sequence variants depends on the length of the variable sequence segment and is calculated as 4̂n, where (n) is the length of the variable segment counted in nucleobases. For example, if the length of the variable segment is four nucleobases, a population comprises 256 oligonucleotides, or 4096 oligonucleotides if the length of the variable segment is six nucleobases.

The variable sequence segment of the oligonucleotides is preferably single-stranded. Such a variable sequence segment allows oligonucleotides of a population of nucleotide conjugates to bind to the single-stranded region of a nucleic acid chain. Such binding is achieved via hybridization of the variable segment of an oligonucleotide in a population of nucleotide-conjugates to a complementary sequence in a nucleic acid chain. Due to the multitude of variable segments present within a population of oligonucleotides, the nucleotide conjugate population can bind to nucleic acid chains of any composition.

Aspect 8: A method for the enzymatic synthesis of nucleic acid chains in which nucleotide conjugates are employed.

Aspect 9: A method for the synthesis of nucleic acid chains, which comprises the following steps:
preparation of template-primer complexes capable of extension
incubation of these complexes in a reaction mixture, which comprises one or more types of polymerase and at least one type of nucleotide conjugate under conditions that permit extension of the primer with nucleotide conjugates, and where each type of nucleotide conjugate bears a characteristic marker.

Aspect 10: A kit for conducting an enzymatic synthesis of nucleic acid chains, which comprise the following elements:
One or more types of polymerases
At lest one type of nucleotide conjugate

Aspect 11A:

A method for sequencing by synthesis of nucleic acid chains comprising the following steps:
a) preparation of at least one population of extendable nucleic acid chain-primer complexes (NAC-primer complexes)
b) incubation of at least one type of nucleotide conjugate and at least one type of polymerase together with the NAC-primer complexes prepared in step (a) under conditions that allow for incorporation of the complementary nuc-components of the nucleotide conjugates, where each type of nucleotide-conjugates possesses a characteristic marker.
c) separation of unincorporated nucleotide conjugates from the NAC-primer complexes
d) detection of the signals of the nucleotide conjugates incorporated into NAC-primer complexes
e) cleaving-off of the linker component as well as of the marker component and oligonucleotide component from the nucleotide conjugates incorporated into the NAC-primer complexes
f) washing of the NAC-primer complexes
   repetition of steps (b) through (f) if required

Aspect 11B:

A method for sequencing nucleic acid chains comprising the following steps:
a) preparation of at least one population of extendable nucleic acid chain-primer complexes (NAC-primer complexes)
b) incubation of at least one type of nucleotide conjugate and at least one type of polymerase together with the NAC-primer complexes prepared in step (a) under conditions that allow for incorporation of the complementary nuc-components of the nucleotide-conjugates, where each type of nucleotide-conjugates possesses a particular characteristic oligonucleotide sequence.
c) separation of unincorporated nucleotide conjugates from the NAC-primer complexes
d) addition of at least one labeled oligonucleotide to the extended NAC-primer complexes and incubation under conditions that allow for specific hybridization of labeled oligonucleotides with the oligonucleotides of the nucleotide conjugates
e) separation of non-hybridized labeled oligonucleotides from the NAC-primer complexes
f) detection of the signals of the nucleotide conjugates incorporated into NAC-primer complexes hybridized with labeled oligonucleotides
g) cleaving-off of the linker component as well as of the marker component and oligonucleotide component from the nucleotide conjugates incorporated into the NAC-primer complexes
h) washing of the NAC-primer complexes
   repetition of steps (b) through (g) if required,

Aspect 11C:

A method for sequencing nucleic acid chains comprising the following steps:
a) preparation of at least one population of extendable nucleic acid chain-primer complexes (NAC-primer complexes)
b) incubation of at least one type of nucleotide conjugates and at least one type of polymerase together with the NAC-primer complexes prepared in step (a) under conditions that allow for incorporation of the complementary nuc-components of the nucleotide-conjugates, where the oligonucleotide of the nucleotide conjugates is not complementary to the nucleic acid chain, and where each type of nucleotide conjugate possesses a particular characteristic marker
c) separation of the unincorporated nucleotide-conjugates from the NAC-primer complexes
d) detection of the signals of the nucleotide conjugates incorporated into NAC primer complexes
e) cleaving-off of the linker component as well as of the marker component and oligonucleotide component from the nucleotide conjugates incorporated into the NAC-primer complexes
f) washing of the NAC-primer complexes
   repetition of steps (b) through (f) if required

Aspect 11D:

A method for sequencing nucleic acid chains comprising the following steps:
a) preparation of at least one population of extendable nucleic acid chain-primer complexes (NAC-primer complexes)
b) incubation of at least one type of nucleotide conjugate and at least one type of polymerase together with the NAC-primer complexes prepared in step (a) under conditions that allow for incorporation of the complementary nuc-components of the nucleotide-conjugates, where at least one segment of the oligonucleotide of nucleotide conjugates is capable of binding to the nucleic acid chain to be sequenced, and where each type of nucleotide conjugate possesses a particular characteristic marker
c) separation of the unincorporated nucleotide-conjugates from the NAC-primer complexes
d) detection of the signals of the nucleotide conjugates incorporated into NAC-primer complexes
e) cleaving-off of the linker component as well as of the marker component and oligonucleotide component from the nucleotide conjugates incorporated into the NAC-primer complexes
f) washing of the NAC-primer complexes
   repetition of steps (b) through (f) if required

Aspect 11E:

A method for sequencing nucleic acid chains comprising the following steps:
a) preparation of at least one population of extendable nucleic acid chain-primer complexes (NAC-primer complexes)
b) incubation of at least four types of nucleotide conjugates and at least one type of polymerase together with the NAC-primer complexes prepared in step (a) under conditions that allow for incorporation of the complementary nuc-components of the nucleotide conjugates, where the oligonucleotide of nucleotide conjugates contains at least one single-stranded segment that is capable of binding to the nucleic acid chain to be sequenced, and where each type of nucleotide conjugate possesses a particular characteristic marker
c) separation of the unincorporated nucleotide-conjugates from the NAC-primer complexes
d) detection of the signals of the nucleotide conjugates incorporated into NAC-primer complexes
e) cleaving-off of the linker component as well as of the marker component and oligonucleotide component from the nucleotide conjugates incorporated into the NAC-primer complexes
f) washing of the NAC-primer complexes
   repetition of steps (b) through (f) if required,

Aspect 11E:

A method for sequencing nucleic acid chains comprising the following steps:
a) preparation of at least one population of extendable nucleic acid chain-primer complexes (NAC-primer complexes)
b) incubation of at least four types of nucleotide conjugates and at least one type of polymerase together with the NAC-primer complexes prepared in step (a) under conditions that allow for incorporation of the complementary nuc-components of the nucleotide conjugates, where each type of nucleotide conjugate is a composition comprising a multitude of nucleotide conjugates, where this composition contains a uniform nucleoside-triphosphate (nuc-component) and many oligonucleotides, where each of said oligonucleotides comprises at least one single-stranded segment and said segments differ in their sequence composition and are capable of binding to the nucleic acid chain to be sequenced, and where each type of nucleotide conjugates possesses a particular characteristic marker c) separation of the unincorporated nucleotide-conjugates from the NAC-primer complexes
d) detection of the signals of the nucleotide conjugates incorporated into NAC-primer complexes
e) cleaving-off of the linker component as well as of the marker component and oligonucleotide component from the nucleotide conjugates incorporated into the NAC-primer complexes
f) washing of the NAC-primer complexes
   repetition of steps (b) through (f) if required,

Aspect 11G: A method for sequencing nucleic acid chains in accordance with the previous aspects of the invention, where the respective composition of nucleotide conjugates comprises oligonucleotides that bind to the nucleic acid chain to be sequenced via single-stranded segments of about 3 to 15 nucleobases.

A further aspect 12 of the invention relates to macromolecular nucleotide compounds according to one of the aspects 1 to 11, wherein the nuc-component comprises the following structures (FIG. 12), wherein:
Base is selected independently from the group of adenine, or 7-deazaadenine, or guanine, or 7-deazaguanine, or thymine, or cytosine, or uracil, or their modifications, wherein (L) is the linkage between the nuc-component and the linker component (coupling unit L) and X is the coupling position of the coupling unit (L) to the base.
R₁—is H
R₂—is selected independently from the group of H, OH, halogen, NH₂, SH or protected OH group
R₃—is selected independently from the group of H, OH, halogen, PO₃, SH, N₃, NH₂, O—R₃₋₁, P(O)ₘ—R₃₋₁ ((m) is 1 or 2), NH—R₃₋₁, S—R₃₋₁, Si—R₃₋₁ wherein R₃₋₁ is a chemically, photochemically or enzymatically cleavable group or comprises one of the following modifications: —CO—Y, —CH₂—O—Y, —CH₂—S—Y, —CH₂—N₃, —CO—O—Y, —CO—S—Y, —CO—NH—Y, —CH₂—CH═CH₂, wherein Y is an alkyl, for instance (CH₂)ₙ—CH₃ wherein n is a number between 0 and 4, or a substituted alkyl, for instance with halogen, hydroxy group, amino group, carboxy group.
R₄—is H or OH
R₅—is selected independently from the group of OH, or a protected OH group, or a monophosphate group, or a diphosphate group, or a triphosphate group, or is an alpha thiotriphosphate group.

A further aspect 13 of the invention relates to macromolecular nucleotide compounds according to one of the aspects 1 to 11, wherein the nuc-component comprises the following structures (FIG. 12), wherein:
Base is selected independently from the group of adenine, or 7-deazaadenine, or guanine, or 7-deazaguanine, or thymine, or cytosine, or uracil, or their modifications capable of enzymatic reactions.
R₁—is H
R₂—is selected independently from the group of H, OH, halogen, NH₂, SH or protected OH group
R₃—is selected independently from the group of O— R₃₋₂-L, P(O)ₘ—R₃₋₂-L and (m) is 1 or 2, NH—R₃₋₂-L, S—R₃₋₂-L, Si—R₃₋₂-L, wherein R₃₋₂ is the coupling position of the linker to the nucleotide and L is the coupling unit (L) of the linker.
R₄—is H or OH
R₅—is selected independently from the group of OH, or a protected OH group, or a monophosphate group, or a diphosphate group, or a triphosphate group, or is an alpha-thiotriphosphate group.

A further aspect 14 of the invention relates to macromolecular nucleotide compounds according to one of the aspects 1 to 11, wherein the nuc-component comprises the following structures (FIG. 12), wherein:
Base is selected independently from the group of adenine, or 7-deazaadenine, or guanine, or 7-deazaguanine, or thymine, or cytosine, or uracil, or their modifications capable of enzymatic reactions.
R₁—is H
R₂—is selected independently from the group of H, OH, halogen, NH₂, SH or protected OH group
R₃—is selected independently from the group of H, OH, halogen, PO₃, SH, NH₂, O—R₃₋₁, P(O)ₘ—R₃₋₁ ((m) is 1 or 2), NH—R₃₋₁, S—R₃₋₁, Si—R₃₋₁ wherein R₃₋₁ is a chemically, photochemically or enzymatically cleavable group.
R₄— is H or OH
R₅— is selected independently from the group of O—R₅₋₁-L, or P—(O)₃—R₅₋₁-L (modified monophosphate group), or P—(O)₃—P—(O)₃—R₅₋₁-L (modified diphosphate group) or P—(O)₃—P—(O)₃—P—(O)₃—R₅₋₁-L (modified triphosphate group), wherein R₅₋₁ is the coupling position of the coupling unit (L) to the nucleotide and coupling unit (L) is a linkage between nuc-component and the linker component.

A further aspect 15 of the invention relates to macromolecular nucleotide compounds according to aspects 12 to 14, wherein the coupling unit (L) comprises the following structural elements:

R₆—NH—R₇, R₆—O—R₇, R₆—S—R₇, R₆—SS—R₇, R₆—CO—NH—R₇, R₆—NH—CO—R₇, R₆—CO—O—R₇, R₆—O—CO—R₇, R₆—CO—S—R₇, R₆—S—CO—R₇, R₆—P(O)₂—R₇, R₆-Si—R₇, R₆—(CH₂)ₙ—R₇, R₆—(CH₂)ₙ—R₇, R₆-A-(CH₂)ₙ—R₇, R₆—(CH₂)ₙ—B—R₇, R₆—(CH═CH—)ₙ—R₇, R₆-(A-CH═CH—)ₙ—R₇, R₆—(CH═CH—B—)ₙ—R₇, R₆-A-CH═CH—(CH₂—)ₙ—R₇, R₆—(—CH═CH—CH₂)ₙ—B—R₇, R₆—(—CH═CH—CH₂—CH₂)ₙ—B—R₇, R₆—(C≡C—)ₙ—R₇, R₆-(A-C≡C—)ₙ—R₇, R₆—(C≡C—B—)ₙ—R₇, R₆-A-C≡C—(CH₂—)ₙ—R₇, R₆—(—C≡C—CH₂)ₙ—B—R₇, R₆—(—C≡C—CH₂—CH₂)ₙ—B—R₇,
wherein R₆ is the nuc-component, R₇ is the rest of the linker, and A and B comprise independently the following structural elements: —NH—, —O—, —S—, —SS—, —CO—NH—, —NH—CO—, —CO—O—, —O—CO—, —CO—S—, —S—CO—, —P(O)₂—, —Si—, —(CH₂)ₙ—, a photolabile group, wherein (n) ranges from 1 to 5,

A further aspect 16 of the invention relates to macromolecular nucleotide compounds according to aspects 12 to 15, wherein the linker-component comprises a hydrophilic, water-soluble polymer.

Aspect 17: A kit for the labeling of nucleic acid chains in accordance with the method of any of aspects which comprises the following components:
one or several types of polymerases
at least one kind of nucleotide analoga (nuc macromolecule) in accordance with aspects 1 to 16
a solid phase for binding of labeled nucleic acid chains

Aspect 18: A kit for the labeling of nucleic acid chains in accordance with the method of any of aspects, which comprises one or several components selected from the following group, provided as a solution in concentrated or deluted form or also as a mixture of dry substances:
one or several types of polymerases
at least one kind of nucleotide analoga (nuc macromolecule) in accordance with aspects 1 to 16
solutions for carrying out enzymatic reactions
composition for incorporation reaction, including at least one of further nucleoside triphosphates
composition for the binding of labeled nucleic acid chains to the solid phase
composition for washing the solid phase after the incorporation reaction
composition for optical detection of the signals on the solid phase

Aspect 19: A kit for the amplification and labeling of nucleic acid chains in accordance with the method of any of aspects, which comprises one or several components selected from the following group:
one or several types of polymerases
one or several primers for amplification of nucleic acid chains
at least one kind of nucleotide analoga (nuc macromolecule) in accordance with aspects 1 to 16
solutions for carrying out enzymatic reactions
composition containing four dNTPs or NTPs
composition for the binding of labeled nucleic acid chains to the solid phase
composition for washing the solid phase after the incorporation reaction
composition for optical detection of the signals on the solid phase

Aspect 20: A kit for the amplification and labeling of nucleic acid chains according to the method of any of aspects, which comprises at least one of the polymerases selected from the following group:
Reverse Transcriptases: M-MLV, RSV, AMV, RAV, MAV, HIV
DNA Polymerasen: Klenow Fragment DNA Polymerase, Klenow Fragment exo minus DNA Polymerase, T7 DNA Polymerase, Sequenase 2, Vent DNA Polymerase, Vent exo minus DNA Polymerase, Deep Vent DNA Polymerase, Deep Vent exo minus DNA Polymerase, Taq DNA Polymerase, Tli DNA Polymerase, Pwo DNA Polymerase, Thermosequenase DNA Polymerase, Pfu DNA Polymerase

Aspect 21: A kit for the labeling of nucleic acid chains according to the method of any of aspects, wherein components of the composition are provided in a premixed form.

### 1.5. Examples of Embodiments

Examples for coupling reactions of individual components of nuc macromolecules are given in patent applications Cherkasov et al WO2011050938, Cherkasov et al WO 2005044836, Cherkasov et al WO2006097320, Cherkasov et al WO 2008043426, Cherkasov et al DE 10356837, Cherkasov et al DE 102004009704. Nuc macromolecules can be purchased from Genovoxx GmbH (custom synthesis).

### Material:

dUTP-AA (dUTP allyl amine, Jena Bioscience), dCTP-PA (dCTP propargyl amine, Jena Bioscience), dATP-PA (7-(3-amine-1-propynyl)-2′ deoxy-7-deazaadenosin-5′-triphosphat) (custom synthesis of Jena Bioscience), dGTP-PA (7-(3-amine-1-propynyl)-2′-deoxy-7-deazaguanosin-5″-triphosphat, (custom synthesis of Jena Bioscience), PDTP (3-(2-pyridinyl-dithio)-propionic acid, Fluka), 7-(3-phthalimido-1-propynyl)-2′-deoxy-7-deazaguanosine and 7-(3-phthalimido-1-propynyl)-2′-deoxy-7-deazaadenosine (Chembiotech), PDTP-NHS (3-(2-pyridinyl-dithio)-propionic acid-N-hydroxysuccinimidyl ester, Sigma), TCEP (tris-(2-carboxyethyl)phosphine, Sigma), J-Ac (iodoacetate, Sigma), iodacetamide (Sigma), Gamma-((6-aminohexyl)-imido)-dUTP (Jena Bioscience).

### List of Suppliers and Companies:

Aldrich—see Sigma
Fluka—see Sigma
Jena Bioscience—Jena Bioscience, Jena, Germany
Molecular Probes—Molecular Probes Europe, Leiden, Netherlands
MWG—MWG Biotech, Ebersberg near Munich, Germany,
Roche—Roche, Mannheim, Germany
Sigma—Sigma-Aldrich-Fluka, Taufkirchen, Germany
Trilink—Trilink Biotechnologies Inc. San Diego, Calif., USA,

Solvents (Fluka) were, where necessary, used in absolute form or dried according to standard procedures. For solvent mixtures, the mixing ratios provided are in respect of volumes used (v/v).

### 1.5.14 Examples of Synthesis of Nuc Macromolecules

There are many known methods for covalent coupling of substances to nucleic acid chains. The labeling can be conducted at different positions of the nucleic acid chain (5 position, 3 position, internal portions). Multiple labels can be attached to one nucleic acid chain. The modification can be conducted via chemical or enzymatic reactions. “Protocols for Oligonuccleotide and Analogs” S. Agrawal, 1993, “Protocols for Oligonucleotide conjugates” S. Agrawal 1994, Y. Singh et al Chem. Soc. Rev. 2010, 39, 2054-. On the one hand, the coupling of a substance can be carried out already during the chemical/enzymatic synthesis of nucleic acids (for example, by the use of phosphoroamidites or by the use of modified nucleotides and a polymerase or by the use of a ligase reaction). On the other hand, the coupling can proceed via one or more intermediate steps such as through the introduction of a reactive group and be accomplished after the synthesis.

Below, examples which describe some of these variants are presented for demonstration.

Synthesis of Nuc Linker Components with Reactive Groups.

The coupling of nuc components and oligonucleotides can be achieved by many methods. For example, many methods are known which describe the linking of two structures each having a reactive amino group by a crosslinker. Oligonucleotides modified with one or more amino groups can be purchased commercially. Optionally, the amino group can be present at the 5′ end or at the 3′ end, or in the internal area of an oligonucleotide. In the following examples, amino-reactive nuc components are described as precursors. Such amino-reactive nucleotides can be linked to the oligonucleotides. Oligonucleotides that have a mercapto group at one of the ends may also be synthesized (e.g. available from Thermo Fisher Scientific, Germany). Other examples of the introduction of reactive groups into oligonucleotides are also known to a person skilled in the art.

### Example 1

### Synthesis of dUTP-AA-PDTP, dGTP-PA-PDTP, dATP-PA-PDTP, dCTP-PA-PDTP

### Synthesis was Conducted Similary as Described in WO 2005 044836

dUTP-AA (20 mg) was dissolved in 1 ml of water and the pH value was adjusted to 8.5 with NaOH. PDTP-NHS (60 mg dissolved in 0.5 ml methanol) was added dropwise to this aqueous solution of dUTP-AA under stirring. The reaction was carried out at 40° C. for 2 hours. The isolation of the product from excess of PDTP-NHS and PDTP was performed on preparative TLC plates. The resulting products, dUTP-AA-PDTP and dUTP-AA remained on the start line. The nucleotides were eluted from the plate with water and dried. This dUTP analog comprises a disulfide bond that can react with other thiols in a thiol exchange reaction and can be cleaved under mild conditions.

Other nucleotide analogs, such as 7-deaza-aminopropargyl-deoxy-guanosine triphosphate, 7-deaza-aminopropargyl deoxy-adenosine triphosphate and 5-amino-propargyl-deoxycytidine triphosphate were modified in the same way, resulting accordingly in dGTP-PA-PDTP, dATP-PA-PDTP, dCTP-PA-PDTP.

### Example 2

### Synthesis of dUTP-PEG(9)-NHS, dUTP-DTBP-NHS and dUTP-tartrate-NHS

### Example 2A

Coupling of a short linker to the base of a nucleotide.

dUTP-AA (aminoallyl-dUTP, 5 mg, Trilink Biotechnologies, pH 7.0), was dried and suspended in dry DMSO up to a calculated concentration of 20 mmol/l. PEG(9)-(NHS)2 (BS(PEG)9 obtained from Thermo Scientific Germany) was dissolved in DMSO to concentrations of 150 mmol/l.

The suspension of dUTP-AA was added to solution of PEG(9)-(NHS)2 and incubated for 2 h at 37° C. under vigorous stirring until the solution became transparent. The conversion of dUTP-AA was monitored by TLC.

The purification of dUTP-PEG(9)-NHS was carried out by precipitation from diethyl ether/DMF mixture (v: v 90:10). The pellet contained the product. The product was dissolved in DMSO and frozen.

In a similar manner, further dUTP-R—X analogs can be synthesized, wherein (R) represents any linker and (X) can be any reactive group. The reactive group can, for example, react with amino groups or thio groups or carboxyl groups. Examples of other commercially available short linkers (cross-linkers) are presented in the cross-linker Guide Thermo Scientific (www.piercenet.com).

Such linkers can also contain a cleavable linkage such as a reductively cleavable bond, for example dithiodispropionic acid-(NHS)2 or an oxidative cleavable bond such as tartrate-(NHS)2. Both linkers were purchaised from Thermo Scientific.

dUTP-DTBP-NHS was obtained by a similar method as dUTP-PEG(9)-NHS. Dithiodispropionic acid-(NHS)₂ (DTBP-(NHS)₂) was used instead of PEG(9)-(NHS)₂. This dUTP-Analog had a linker containing a disulfide bond, that can be reductively cleaved for example by TCEP.

dUTP-tartrate-NHS was obtained by a similar method as dUTP-PEG(9)-NHS. Tartrate-(NHS)₂ was used instead of PEG(9)-(NHS)₂. This nucleotide had a linker containing a diol bond (—CH2(OH)—CH2(OH)—), that can be oxidatively cleaved for example by KClO4.

The NHS group of the linker can react with an amino group of another molecule, for example with one of an oligonucleotide.

### Example 2B

Coupling of a short linker to the gamma-phosphate group of a nucleotide.

Coupling of PEG(9)-(NHS)2 or Dithiobispropionic acid-(NHS)2 to the amino group of the gamma-((6-aminohexyl)-imido)-dUTP was carried out under similar conditions. The resulting derivatives of dUTP carry a reactive NHS-group bound to gamma phosphate residue: NHS-PEG(9)-ppp-dUTP and NHS-DTBP-ppp-dUTP.

### Examples of Oligonucleotides Modifications.

Oligonucleotides with an amino group at either end can be modified, for example, with active esters (such as NHS ester).

### Example 3

Synthesis of PDTP-Oligo 1
Oligo 1:
5'-NH2-taatacgactcactatagg-3' phosphate

Oligo 1 was modified by means of excess of PDTP-NHS in phosphate buffer/DMSO (20% DMSO), pH 8, with the effect of introducing a disulfide group at the 5′-end of oligo 1 (PDTP-oligo 1). The modified oligonucleotide was purified by means of DEAE chromatography.

### Coupling of Nuc-Components to Oligonucleotide.

### Example 4

### Synthesis of dUTP-AA-SS-Oligo 1, dATP-PA-SS-Oligo 1, dGTP-PA-SS-Oligo1, dCTP-PA-SS-Oligo1 by formation of a disulfide bond

Synthesis of dUTP-AA-SS-Oligo 1.

dUTP-AA-PDTP (ten equivalents) was added to PDTP-oligo 1 (one equivalent, 1 mmol/l) in buffer solution. TCEP was added to this solution (up to a final concentration of 10 mmol/l) to achieve reduction of disulfide groups and formation of dUTP-AA-SH and SH-oligo 1. Next, iodine-solution (saturated solution of I2 in KI solution) was added to the reaction mixture until the yellow color of I2 remained visible. Addition of iodine led to formation of disulfide bridges by oxidation. The product was purified on DEAE column.

dATP-PA-SS-Oligo1, dGTP-PA-SS-Oligo1, and dCTP-PA-SS-Oligo1 were obtained in a similar manner by using dGTP-PA-PDTP, dATP-PA-PDTP, dCTP-PA-PDTP instead of dUTP-AA-PDTP.

### Example 5

### Synthesis of dUTP-PEG(9)-Oligo 2-fluorescein, dUTP-AA-SS-Oligo2-Fluorescein

Oligo-2 Fluorescein,
5′NH2-cgt att acc gcg gct gct gg cac AAAAAAAAAA
FAM

An amino-group (NH2) is bound to the 5′-end via C6 linker and a dye (fluorescein) is coupled to the 3′-end (see list of sequences):

The oligonucleotide was dissolved in phosphate buffer (pH 8.0) to obtain a concentration of 1 mmol/l. A 5-fold excess of dUTP-PEG(9)-NHS, dissolved in DMSO, was added to this solution. The reaction proceeded at room temperature. The subsequent purification of the product was carried out on DEAE column and RP-C18 column. The product, dUTP-PEG(9)-Oligo2-Fluorescein, was dried and subsequently dissolved in water to obtain a concentration of 50 μmol/l, and frozen.

dUTP-AA-SS-Oligo2-Fluorescein was synthesized in a similar manner by using dUTP-DTBP-NHS in place of dUTP-PEG(9)-NHS.

dUTP-ppp-SS-Oligo2-Fluorescein was likewise synthesized by using NHS-DTBP-ppp-dUTP instead of dUTP-PEG(9)-NHS. Likewise, dUTP-ppp-PEG(9)-Oligo2-Fluorescein was synthesized in a similar manner by using the NHS-PEG(9)-ppp-dUTP in place of dUTP-PEG(9)-NHS. In these nucleotide conjugates, oligonucleotides are coupled to the terminal phosphate group of the nuc-component via a linker.

### Example 6

### Synthesis of dUTP-AA-SS-Oligo4-Fluorescein

(Oligonucleotide with Self-Complementary Sequences of the Type “Molecular Beacon”)

A modified oligonucleotide containing self-complementary sequence segments (oligo 4) was purchased (custom synthesis, Eurofins MWG, Germany). An oligonucleotide of this composition may be present in solution as a fully or partially double-stranded oligonucleotide, also known as a “molecular beacon”.

Oligo-4, Fluorescein,
5′NH2-cgt att acc gcg gct gct GTAATAC AAAAA AAAAA
FAM
(Stem regions are underlined)

dUTP-DTBP-NHS was chosen as a nuc-component with a linker (synthesis see above). The oligonucleotide was dissolved in a phosphate buffer solution (pH 8), resulting in a concentration of 1 mmol/l, and added to 5 equivalents of dUTP-DTBP-NHS (dissolved in DMSO). The reaction proceeded at the NH2-group at the 5′-end with good yield. The product was purified on DEAE column and RP-18 column.

### Example 7

### Synthesis of dUTP-AA-SS-Oligo 2-Fluorescein/Oligo 3

Initially, dUTP-AA-SS-Oligo2-Fluorescein was synthesized (see above) and dissolved in a buffer solution. One equivalent of a complementary oligonucleotide with the following structure was added to this solution:

Oligo-3,
5′gtg cc agc agc cgc ggt aat acg 3′phosphate

Oligo 3 can undergo complementary binding with the sequence of oligo 2, thereby blocking part of oligo2 at 5′-end (underlined)

5′NH2-cgt att acc gcg gct gct gg cac AAAAAAAAAA
Fluorescein 3′Phosphate gca taa tgg cgc cga cga
cc gtg

The solution containing dUTP-AA-SS-Oligo2-Fluorescein and oligo 3 (50 mmol/l Tris-HCl, pH 8.0) was heated at 90° C. for 1 minute, and then cooled to RT. The two complementary sequence segments bind to each other during cooling to form a double strand.

### EXAMPLE

### Cleaving of a Cleavable Group in the Linker and Blocking of Free SH-Group if Required

The following conditions may be used for reductive cleavage of a disulfide bond: TCEP 10 to 50 mmol/l, pH 6.0 to 8.0, at room temperature for about 5 to 30 minutes. Cleavage conditions of this type are suitable for nucleotide conjugates with a cleavable linker containing dithiobispropionic acid for example.

For oxidative cleavage of linkers containing a diol-bond (e.g. tartrate linker) the following conditions may be used: KCI04 5 to 50 mmol/l, pH 6.0 to 8.0, at room temperature for about 5 to 20 min.

A free SH-group present after cleavage of a disulfide bond may be blocked, for example, by means of iodoacetamide: 0.1 to 0.5 mol/l iodoacetamide in a buffer with pH 7.0 to 8.0 for about 5 to 15 min at RT.

### Enzymatic Incorporation of Nucleotide Conjugates:

### Example 9

Enzymatic incorporation reactions were carried out at customary conditions for incorporation reactions for modified nuc-macromolecules. The following conditions may be used for example:

### Buffer Solutions:

Tris-HCl (20 mM-100 mM), pH 7-8.5
MgCl₂ e.g. 1.5 to 10 mM (or Mn 0.2-1 mM)
NaCl 10 to 100 mM
Glycerin ca. 10-30%
DMSO ca. 5 to 30%
Primers (oligonucleotides) of a length of 17 to 50 nucleotides that exhibit sufficiently specific hybridization to the template.
Concentration approximately from 0.02 to 2 μM.
Templates (e.g. oligonucleotides).
DNA polymerases (Klenow fragment exo minus, Vent exo minus polymerase).
Nucleotide conjugates are preferably used in concentrations of 0.1 μmol/l to 10 μmol/l.

Enzymatic reactions were carried out for approx. 2 to 60 min at temperatures between room temperature and up to 60° C.

### Example 9

### Materials

Reaction buffer 1: 50 mmol/l Tris HCl, pH 8.5; 50 mmol/l NaCl, 5 mmol/l MgCl₂, Glycerin 10% v/v
Reaction buffer 2: 10 mmol/I Tris HCl, pH 8.5; 10 mmol/I NaCl, 1 mmol/l MgCl₂, Glycerin 2%, DMSO 20% v/v

### Example 10

Preparation of a modified exo minus Klenow fragment of DNA polymerase I of E. coli (hereafter referred to as modified Klenow Exo-minus). In one implementation of this modification, 100 μl of Tris-buffer solution (200 mmol/l, Tris-HCl buffer, pH 9.0, 60% glycerol) was added to 70 μl buffer solution containing Klenow exo minus DNA polymerase (New England Biolabs), whereupon the pH-value of polymerase solution was 8.5-9.0. Next, iodacetamide (20 μl, dissolved in water to 1 mol/l) was added. The reaction took place for 5 min at RT. In this manner, a selective modification of the polymerase at the SH-group of cysteine was achieved, and the DTT in the manufacturer's buffer was inactivated. The modified polymerase was stored at −20° C.

### Example 11

### Enzymatic Incorporation and Termination of Synthesis by dUTP-AA-SS-Oligo1, dATP-PA-SS-Oligo1, dGTP-PA-SS-Oligo1, dCTP-PA-SS-Oligo1

Reversible termination of synthesis at a homopolymeric sequence segment presents a particular challenge for sequencing-by-synthesis methods. The ability of nucleotide analogs to reversibly block the enzymatic reaction after their incorporation is demonstrated by using templates with homopolymeric sequence segments as an example.

Nucleotide conjugates were used in concentrations of 2 μmol/l and 0.2 μmol/l (as described in the legend). Modified Klenow exo minus fragment was used (1 unit/20 μl reaction mixture). The primer T7-19-Cy was used at a concentration of 1 μmol/l. Oligonucleotides (1 μmol/l) that permit single or multiple incorporation of the corresponding complementary nucleotide conjugates were used as templates. Natural substrates (dNTPs, 200 μmol/l) were added to some of the reactions, as indicated in the legend.

In reaction buffer 1, the reaction proceeded at 37° C. for 1 hr. The reaction mixture was subsequently applied to a 10% polyacrylamide gel, and the reaction products were separated at 150 V (70° C.). The visualization was performed with the help of a gel documentation apparatus with UV light source.

### Legend:

Lane 1: dATP-PA-SS-Oligo1 (2 μmol/l), Template 4
Lane 2: dATP-PA-SS-Oligo1 (0.2 μmol/l), Template 4
Lane 3: dATP-PA-SS-Oligo1 (2 μmol/l), Template 5
Lane 4: dATP-PA-SS-Oligo1 (0.2 μmol/l), Template 5
Lane 5: dCTP-PA-SS-Oligo1 (2 μmol/l), Template 6
Lane 6: dCTP-PA-SS-Oligo1 (0.2 μmol/l) Template 6
Lane 7: dCTP-PA-SS-Oligo1 (2 μmol/l), Template 7
Lane 8: dCTP-PA-SS-Oligo1 (0.2 μmol/l), Template 7
Lane 9: dGTP-PA-SS-Oligo1 (2 μmol/l), Template 5, dATP, dCTP
Lane 10: dGTP-PA-SS-Oligo1 (0.2 μmol/l), Template 5, dATP, dCTP
Lane 11: dGTP-PA-SS-Oligo1 (2 μmol/l), Template 8, dATP
Lane 12: dGTP-PA-SS-Oligo1 (0.2 μmol/l), Template 8, dATP
Lane 13: dUTP-AA-SS-Oligo1 (2 μmol/l), Template 2
Lane 14: dUTP-AA-SS-Oligo1 (0.2 μmol/l), Template 2
Lane 15: dUTP-AA-SS-Oligo1 (2 μmol/l), Template 3

The nucleotide analogs that were used here have free 3′-OH groups. One might expect that further nucleotides would be coupled to these groups by the polymerase. FIG. 13 shows that only a single nucleotide conjugate was incorporated at all templates (arrow A). Arrow (B) indicates the position of the labeled primer.

dATP-PA-SS-Oligo1 is incorporated only once (Lane 1) at template 4 (homopolymer segment). Following incorporation of dATP-PA-SS-Oligo1, the incorporation of further dATP-PA-SS-Oligo1 at the adjacent complementary position on the template (N+1) was inhibited. An incorporation reaction with template 5 served as a control. Only one dATP-PA-SS-Oligo1 could be incorporated (lane 3) in this reaction, as the template offers no additional complementary positions for further incorporation of dATP-PA-SS-Oligo1. As a further control, incorporation of dATP-PA-SS-Oligo1 at templates 4 and 5 was carried out at limiting substrate concentrations (0.2 μmol/l dATP-PA-SS-Oligo1 and 1 μmol/l T7-19-Cy3 and template) (lane 2 and 4).

dCTP-PA-SS-Oligo1 is incorporated only once at template 6 (homopolymer segment) (Lane 5). Incorporation of a dCTP-PA-SS-Oligo1 blocked the incorporation of an additional dCTP-PA-SS-Oligo1 at the adjacent position. An incorporation reaction with template 7 served as a control. In this reaction, only one dCTP-PA-SS-Oligo1 could be incorporated (lane 7), as the template offers no further complementary positions for incorporation of dCTP-PA-SS-Oligo1. As a further control, incorporation of dCTP-PA-SS-Oligo1 at templates 6 and 7 was carried out at limiting substrate concentrations (0.2 μmol/l dCTP-PA-SS-Oligo1 and 1 μmol/l T7-19-Cy3 and templates) (lane 6 and 8).

dGTP-PA-SS-Oligo1 is incorporated only once at templates 5 and 8 (lane 9 and 11). Template 5 contains the sequence -CGC-, and template 8 comprises the sequence -CTC-. Hence, both template sequences contain nonadjacent positions for incorporation of dG-analogs. Nevertheless, incorporation of one dGTP-PA-SS-Oligo1 resulted in blockage of the incorporation of a second dGTP-PA-SS-Oligo1 at position N+2. Incorporation of dGTP-PA-SS-Oligo1 at templates 5 and 8 was carried out at limiting substrate concentrations as a control (0.2 μmol/l dGTP-PA-SS-Oligo1 and 1 μmol/l T7-19-Cy3 and template) (lane 10 and 12).

dUTP-AA-SS-Oligo1 is incorporated only once at template 2 (homopolymer segment) (lane 13). The incorporation of dUTP-AA-SS-Oligo1 resulted in blockage of the incorporation of another dUTP-AA-SS-Oligo1 at the adjacent position. An incorporation reaction at template 3 was used as control. In this reaction, only one dUTP-AA-SS-Oligo1 could be incorporated (Lane 15), as the template provides no further complementary position for incorporation of dUTP-AA-SS-Oligo1. As a further control, the incorporation of dUTP-AA-SS-Oligo1 at template 2 was carried out at limiting substrate concentrations (0.2 μmol/l dUTP-AA-SS-Oligo1 and 1 μmol/l T7-19-Cy3) (lane 14).

This demonstrates that only a single nucleotide conjugate was incorporated at homopolymeric stretches of the template, the incorporation of further nucleotide conjugates of the same type was inhibited. This inhibition was observed at the adjacent position (N+1), as well as at subsequent positions (N+2). The analogs used here can thus act as terminators of synthesis. The presence of a disulfide bridge in the linker of the nucleotide conjugates makes it possible to cleave the oligonucleotide moieties from the incorporated nuc-macromolecules.

After the oligonucleotide moiety of any incorporated nucleotide conjugate(s) has been cleaved off, and after the free group has been blocked by means of iodoacetamide, a further nucleotide conjugate (n+1) can be incorporated.

In an advantageous embodiment, all four types of nucleotide conjugates (e.g. dATP-conjugates, dCTP-conjugates, dGTP-conjugates and dUTP-conjugates) may be used simultaneously in a single reaction. Preferably, no natural nucleotides (such as dNTPs) are added to the reaction.

The templates can be attached to a solid phase. Such a reaction is often carried out in cyclic mode, which means that the template can be washed between individual reaction steps.

Instead of dithiobispropionic acid linker, other linkers of similar length, such as tartrate linker, or longer linkers, such as PEG (9), may be used. Such nucleotide conjugates also exhibit terminating or reversibly terminating properties.

### Example 12

### Incorporation of Nucleotide Conjugates with a Double-Stranded Sequence Segment dUTP-AA-SS-Oligo 4-Fluorescein (Oligonucleotide Containing Self-Complementary Sequences of the Type “Molecular Beacon”) and dUTP-AA-SS-Oligo 2-Fluorescein/Oligo 3

Nucleotide conjugates were used at a concentration of 1 μmol/l. Modified Klenow exo minus was used as the polymerase (1 unit/20 μl reaction mixture). T7-19-Cy was used as a primer (1 or 0.2 μmol/l). Oligonucleotides (1 or 0.2 μmol/l) that allow for incorporation of a single (template 3) or of multiple (template 2) correspondingly complementary nucleotide conjugates were used as templates. The reaction proceeded in reaction buffer 1 or reaction buffer 2 at 37° C. for 1 hr. The reaction mixture was subsequently analyzed by means of capillary electrophoresis (ABI 310 capillary sequencer, POP6 gel matrix). Electrophoresis was performed at 12 kV and 50° C. The signals of Cy3-dye as well as fluorescein dye were detected. CE-profiles are shown in FIGS. 14-21.

FIG. 14 T7-19-Cy3 primer only (control)

FIG. 15 dUTP-AA-SS-Oligo 2-fluorescein/Oligo 3 only

FIG. 16 dUTP-AA-SS-Oligo 4-fluorescein only

FIG. 17 B1: dUTP-AA-SS-Oligo 4-Fluorescein, modified Klenow Exo minus, Template 2 (1 μmol/l), Primer T7-19-Cy3 (1 μmol/l), Reaction buffer 1

FIG. 17 B3: dUTP-AA-SS-Oligo 4-Fluorescein, modified Klenow Exo minus, Template 2 (0.2 μmol/l), Primer T7-19-Cy3 (0.2 μmol/l), Reaction buffer 1

FIG. 18 B5: dUTP-AA-SS-Oligo 4-Fluorescein, modified Klenow Exo minus, Template 2 (0.2 μmol/l), Primer T7-19-Cy3 (0.2 μmol/l), Reaction buffer 2

FIG. 18 B6: dUTP-AA-SS-Oligo 4-Fluorescein, modified Klenow Exo minus, Template 3 (0.2 μmol/l), Primer T7-19-Cy3 (0.2 μmol/l), Reaction buffer 2

FIG. 19 C1: dUTP-AA-SS-Oligo 2-Fluorescein/Oligo 3, modified Klenow Exo minus, Template 2 (1 μmol/l), Primer T7-19-Cy3 (1 μmol/l), Reaction buffer 1

FIG. 20 C3: dUTP-AA-SS-Oligo 2-Fluorescein/Oligo 3, modified Klenow Exo minus, Template 2 (0.2 μmol/l), Primer T7-19-Cy3 (0.2 μmol/l), Reaction buffer 1

FIG. 21 C5: dUTP-AA-SS-Oligo 2-Fluorescein/Oligo 3, modified Klenow Exo minus, Template 2 (0.2 μmol/l), Primer T7-19-Cy3 (0.2 μmol/l), Reaction buffer 2

The nucleotide analogs that were used here have free 3′-OH groups. One might expect that further nucleotides would be coupled to these groups by the polymerase. Arrow (A) indicates the position of the labeled primer and of unincorporated nucleotide conjugates. This demonstrates that both nucleotide conjugates used are incorporated only once at a homopolymer stretch (template 2) (arrow B). Controls: primer and nucleotide conjugates alone, single incorporation event of dUTP-AA-SS-Oligo 4-Fluorescein at template 3.

### Example 13

### Composition of a Kit for the Use of Nucleotide Conjugates

Generally, one or more kits comprise components (e.g. individual substances, compositions, reaction mixtures) that are required for the implementation of enzymatic incorporation reactions with modified nuc-macromolecules according to the present invention.

The composition of the kit may vary depending on the application, where the application can range from a simple primer extension reaction to cyclic sequencing at the single-molecule level.

Kits for cyclic sequencing may for example comprise polymerases, modified nuc-macromolecules, as well as solutions for the cyclic steps.

Optionally, kits may contain positive and/or negative controls, instructions for performing procedures.

The kit components are generally provided in customary reaction vessels, where the volume of the vessels may range from 0.2 ml to 11. Alternatively, vessel arrays, such as microtiter plates, my be loaded with components, which supports the automatic addition of reagents.

A kit may comprise the following components:
One or more polymerases, such as modified Klenow fragment exo minus
One or more types or one or several populations of nucleotide conjugates, which may be present in the form of an acid or a salt (e.g. sodium, potassium, ammonium, or lithium ions may be used). The nucleotide conjugates can be provided as dry substances or in solution, for example in water or in a buffer, such as Tris-HCl, HEPES, borate, phosphate, acetate, or in a storage solution, which may comprise the following components, individually or in combination:
   buffer Tris-HCl, HEPES, borate, phosphate, acetate (at concentrations between 10 mM and 200 mM for example)
   salts such as NaCl, KCl, NH₄Cl, MgCl₂,
   PEG or other inert polymers, such as mowiol at a concentration of 1 to 20% (w/v)
   glycerol at concentrations between 1% and 50%
   marker or marker units of modified nuc-macromolecules, particularly in embodiments wherein affinity coupling is present between the linker and the marker or between the marker and the core-component.
Buffer compositions for enzymatic reaction, cleavage, blockade, detection, washing steps:
   Cleavage reagents, provided, for example, as a concentrated buffered solution. E.g. DTT or TCEP in embodiments with nucleotide conjugates containing a linker with a cleavable disulfide bond.
   Modifying reagents, provided, for example, as a concentrated buffered solution. E.g. iodoacetamide or iodoacetate for embodiments where the linker carries a mercapto-group after cleavage.
   Detection reagents, such as labeled oligonucleotides which can be hybridized to nucleotide conjugates.

### List of Sequences:

Primer
Primer T7-19-Cy3: 5′-Cy3- taatacgactcactatagg

### Examples of Oligonucleotide Components of the Nucleotide Conjugates

Oligo 1
5'-NH2-taatacgactcactatagg-3' phosphate

This oligonucleotide may be used in combination with the following nuc-components for example:

Coupling of a PEG-linker to the base:
dUTP-PEG(9)-taatacgactcactatagg
Coupling of a cleavable linker to the base:
dUTP-AA-SS-taatacgactcactatagg
Coupling of a PEG linker to the gamma-phosphate
group:
dUTP-ppp-PEG(9)-taatacgactcactatagg
Coupling of a cleavable linker to the gamma-
phosphate group:
dUTP-ppp-SS-taatacgactcactatagg

Oligonucleotide moiety may serve as a characteristic marker sequence for dUTP. Another characteristic sequence can be used for another nuc-component.

A part of this oligonucleotide can serve as a binding segment (B segment).

Oligo2, fluorescein,
5′NH2-cgt att acc gcg gct gct gg cac AAAAAAAAAA
3′- fluorescein

The homopolymeric portion of this oligonucleotide (AAAAAAAAAA) provides an example of a variable segment. It can bind to a portion of another nucleic acid chain that contains several thymidine residues (e.g. TTTTTT). A loose, transient interaction occurs under reaction conditions, such as reaction buffer 1 or 2 and room temperature or 37° C., since the Tm of (AAAAAAAAAAA) lies below 25° C. Sequence specificity is very low.

This oligonucleotide may be used, for example, in the following combinations with nuc-components:

Coupling of a PEG-linker to the base:
dUTP-PEG(9)- cgt att acc gcg gct gct gg cac
AAAAAAAAAA
Coupling of a cleavable linker to the base:
dUTP-AA-SS- cgt att acc gcg gct gct gg cac
AAAAAAAAAA
Coupling of a PEG-linker to the gamma-phosphate
group:
dUTP-ppp-PEG(9)- cgt att acc gcg gct gct gg cac
AAAAAAAAAA
Coupling of a cleavable linker to the gamma-
phosphate group:
dUTP-ppp-SS- cgt att acc gcg gct gct gg cac
AAAAAAAAAA
Oligo-3,
5′gtg cc agc agc cgc ggt aat acg 3′phosphate

This oligonucleotide undergoes sequence-specific binding with a sequence fragment of oligo 2. The Tm of this oligonucleotide is approximately 70° C. (measured in reaction buffer 1). After hybridization of this oligonucleotide to oligo 2, it remain bound to oligo 2 under reaction conditions (room temperature or 37° C.) and prevents any interactions between oligo 2 and another sequence that is predominantly complementary to oligo2.

This oligonucleotide may contain other modifications, such as fluorescent dyes. When dyes with an excitation spectrum similar to that of rhodamine are used, FRET can be achieved between fluorescein and rhodamine.

Oligo 4, fluorescein,
5′NH2-cgt att acc gcg gct gct GTAATAC AAAAA AAAAA
3′-fluorescein
(Stem regions are underlined)

This oligonucleotide contains self-complementary sequences (underlined) and, under reaction conditions (reaction buffer 1 or 2, RT or 37° C.), predominantly adopts the form of a molecular beacon. The interaction of this sequence segment with a further nucleic acid sequence complementary to this segment is thereby blocked or greatly reduced. The homopolymeric region of this oligonucleotide (AAAAAAAAAA) can bind to a region of another nucleic acid chain that contains several thymidine residues (e.g. TTTTTT or TTTTTTTTTT). Only a loose, transient interaction occurs under reaction conditions, such as reaction buffer 1 or 2 and at room temperature or 37° C., since the Tm of AAAAAAAAAAA lies below 25° C.

This oligonucleotide may be used, for example, in the following combinations with nuc-components:

Coupling of a PEG linker to the base:
dUTP-PEG(9)- cgt att acc gcg gct gct GTAATAC AAAAA
AAAAA
Coupling of a cleavable linker to the base:
dUTP-AA-SS- cgt att acc gcg gct gct GTAATAC AAAAA
AAAAA
Coupling of a PEG linker to gamma-phosphate group:
dUTP-ppp-PEG(9)- cgt att acc gcg gct gct GTAATAC
AAAAA AAAAA
Coupling of a cleavable linker to gamma-phosphate
group:
dUTP-ppp-SS- cgt att acc gcg gct gct GTAATAC AAAAA
AAAAA

Composition oligo 5 (4096 oligonucleotides containing a uniform sequence region, which is underlined, and a variable, randomized sequence region (X) with a length of 6 NT)

5′NH2-(X)n-cgt att acc gcg gct gct gta
cacAAAAAAAAAA-Fluorescein
(X) = A,C,G,T; (n) = 6

After hybridization of oligo 3 to the oligonucleotides of this population, the underlined fragment of the sequence can be excluded from interactions with other single stranded nucleic acids. Only the variable segment (X)n (a hexamer segment) and AAAAAAAAAA are available for interactions with other nucleic acid chains. As the binding of hexamers to single-stranded nucleic acid chains is unstable under specified reaction conditions (room temperature to 37° C., reaction buffer 1 or 2), only transient binding of nucleotide conjugates to nucleic acid sequences ensues. The sequence specificity of binding of such oligonucleotides is very low owing to the shortness of the variable segment.

This composition of oligonucleotides may be used in the following combinations with nuc-components for example:

Coupling of a PEG linker to the base:
dUTP-PEG(9)- (X)n-cgt att acc gcg gct gct gg
cacAAAAAAAAAA
Coupling of a cleavable linker to the base:
dUTP-AA-SS- (X)n-cgt att acc gcg gct gct gg
cacAAAAAAAAAA
Coupling of a PEG linker to gamma-phosphate group:
dUTP-ppp-PEG(9)- (X)n-cgt att acc gcg gct gct gg
cacAAAAAAAAAA
Coupling of a cleavable linker to gamma-phosphate
group:
dUTP-ppp-SS- (X)n-cgt att acc gcg gct gct gg
cacAAAAAAAAAA

Sequence segment (cgt att acc gcg gct gct gg cac) may serve as a unique characteristic marker sequence, to which sequence-specifically labeled oligonucleotides may be bound.

Composition Oligo 6 (4096 oligonucleotides containing a uniform underlined sequence segment and a variable sequence segment (X) with a length of 6 NT)

5′NH2-TTTTTTTTTTcgt att acc gcg gct gct gg cac-
(X)n-Fluorescein
(X) = A,C,G,T; (n) = 6

Composition of oligo 6 differs from the composition of oligo 5 by virtue of the arrangement of individual sequence segments. Through changes in this arrangement, the nuc-component can be placed at a particular distance from a particular segment of the oligonucleotide. In this example, nuc-components are closer to the homopolymer stretch (TTTTTTTTTT).

This composition of oligonucleotides may be used in the following combinations with nuc-components for example:

Coupling of a PEG linker to the base:
dUTP-PEG(9)- TTTTTTTTTTcgt att acc gcg gct gct gg cac-(X)n
Coupling of a cleavable linker to the base:
dUTP-AA-SS- TTTTTTTTTTcgt att acc gcg gct gct gg cac-(X)n
Coupling of a PEG linker to gamma-phosphate group:
dUTP-ppp-PEG(9)- TTTTTTTTTTcgt att acc gcg gct gct gg cac-(X)n
Coupling of a cleavable linker to gamma-phosphate group:
dUTP-ppp-SS- TTTTTTTTTTcgt att acc gcg gct gct gg cac-(X)n

### Templates:

Template 1 (M1):
5′GTT TTC CCA GTC ACG ACG GGAG gtg cc agc
agc cgc ggt aat acg ACCA cctatagtgagtcgtatta
Template 2 (M2):
5′AAAAAAcctatagtgagtcgtatta3′-phosphate
Template 3 (M3):
5′Acctatagtgagtcgtatta-3′-phosphate
Template 4 (M4):
5′TTTTTcctatagtgagtcgtatta-3′-phosphate
Template 5 (M5):
5′CGCTTTGTcctatagtgagtcgtatta
Template 6 (M6):
5′AGGGcctatagtgagtcgtatta-3′-phosphate
Template 7 (M7):
5′Gcctatagtgagtcgtatta-3′-phosphate
Template 8 (M8):
5′ACTCTcctatagtgagtcgtatta-3′-phosphate

(Primer binding site for T7-19 primers and the relevant positions for incorporation of nucleotide conjugates on the template are stated)

All publications, patents and patent applications cited herein are incorporated into this application in full extent (even if this was not stated explicitly for any particular publication) and, according to the USPTO, are subject to regulations for “incorporated by reference” for all purposes in the U.S.

Particular embodiments were described to illustrate the nature of the invention. They can be further combined with one another by a person skilled in the art. Combinations of various embodiments are also object of the present invention.

### LEGENDS TO FIGURES

FIG. 1

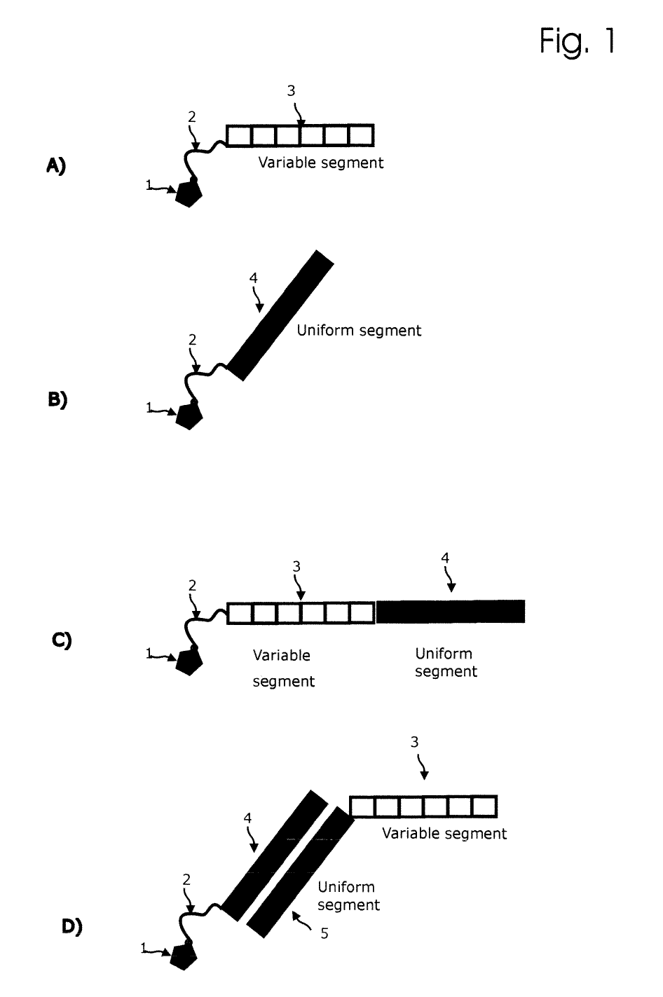

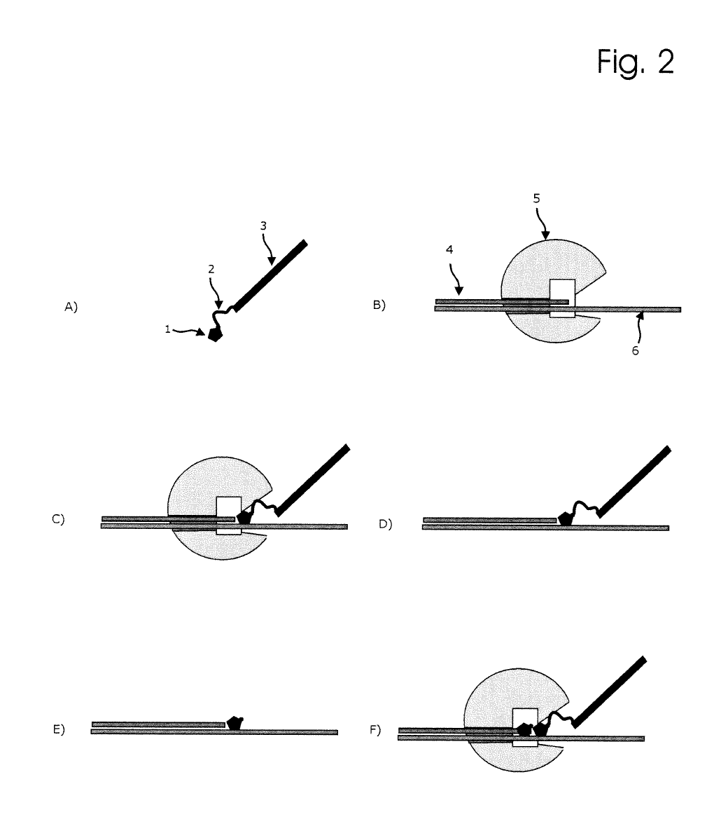

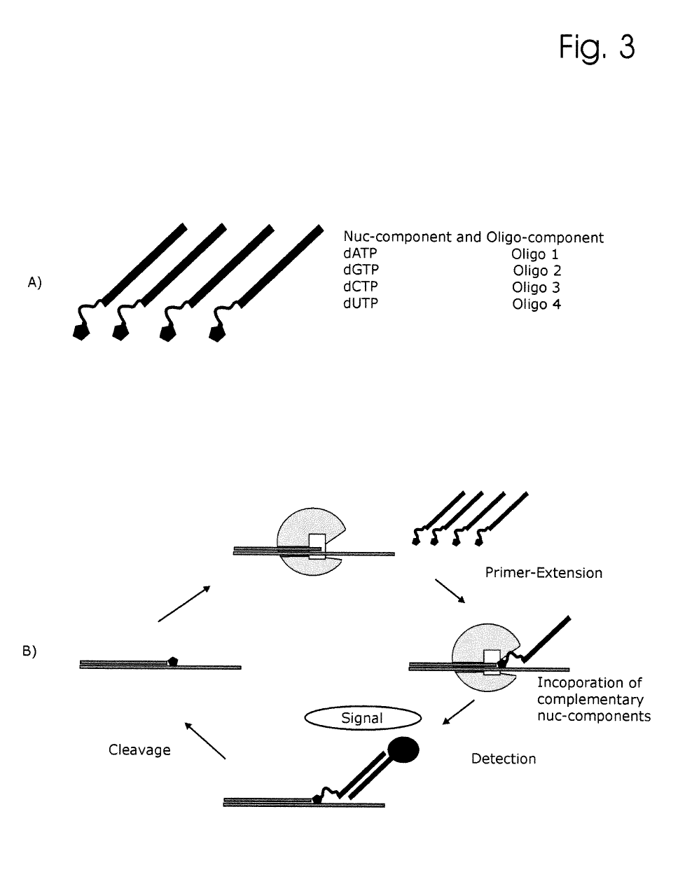

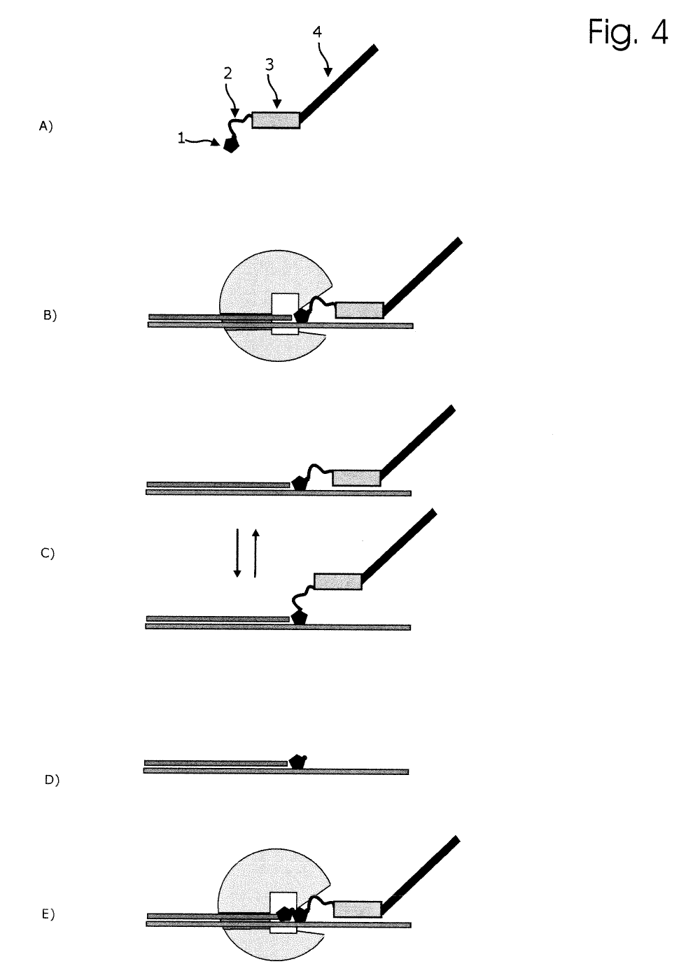

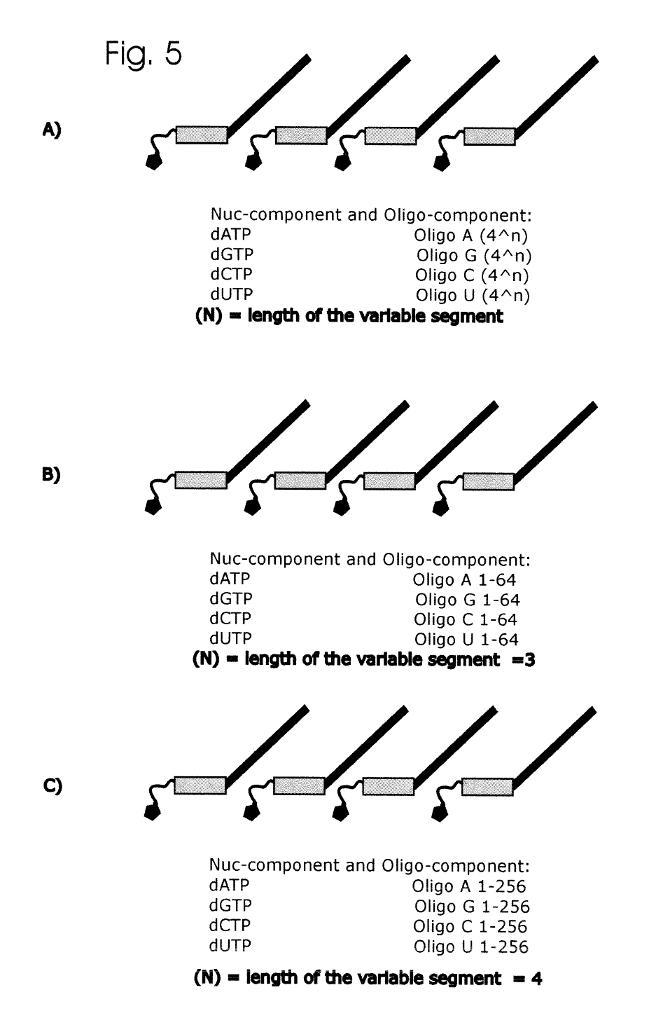

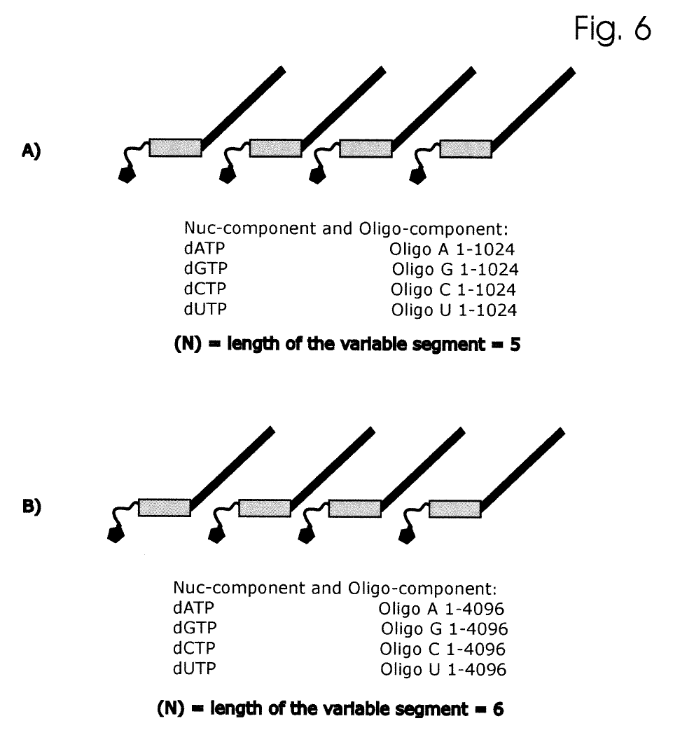

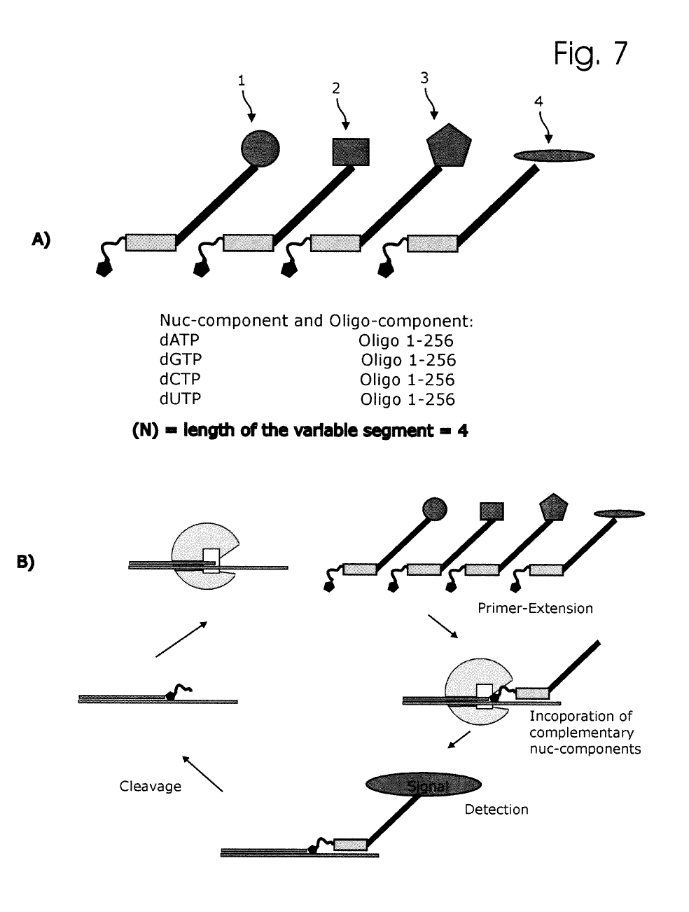

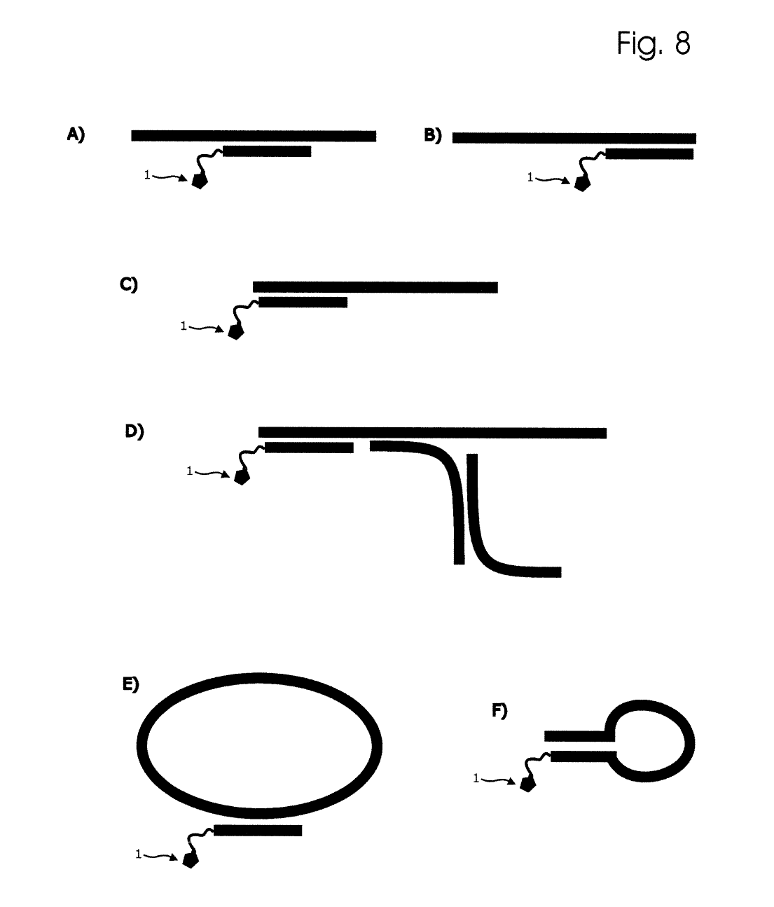

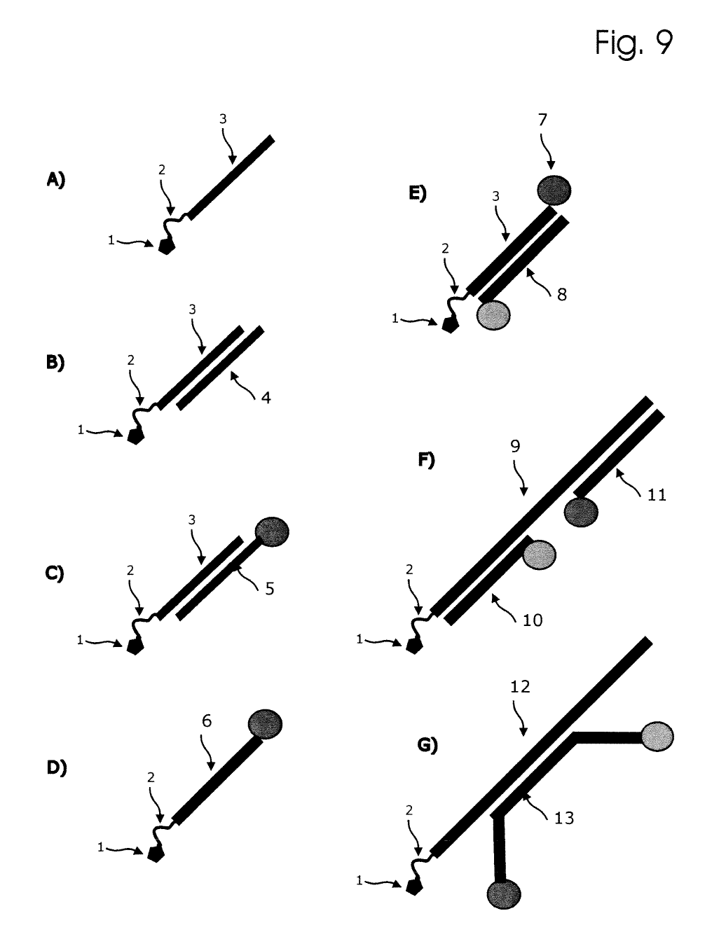

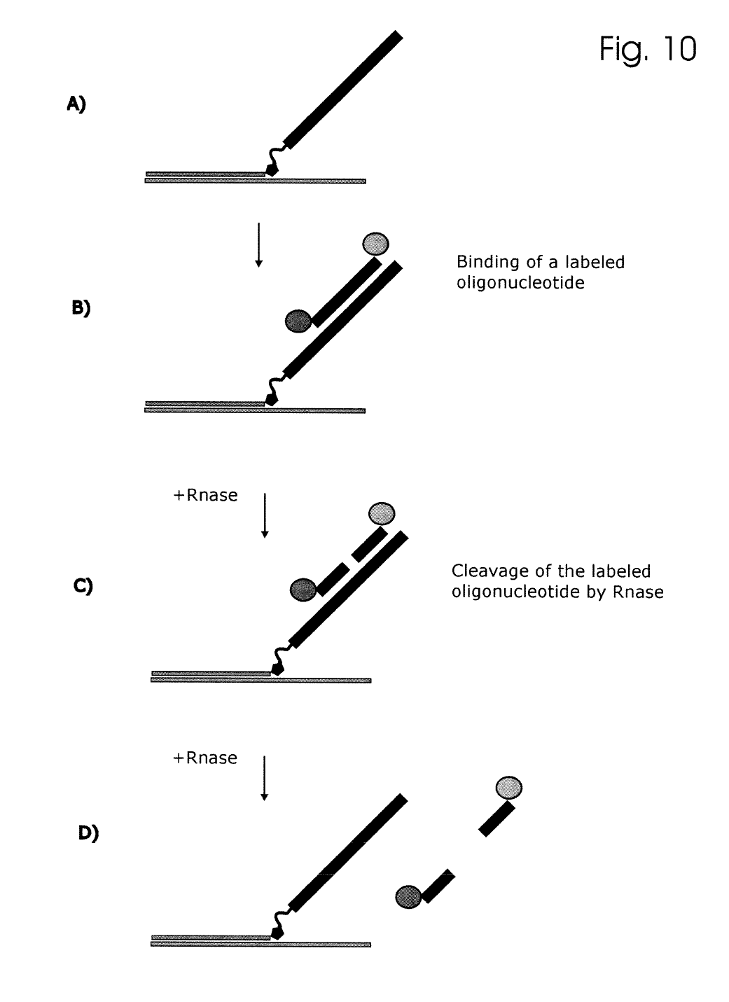

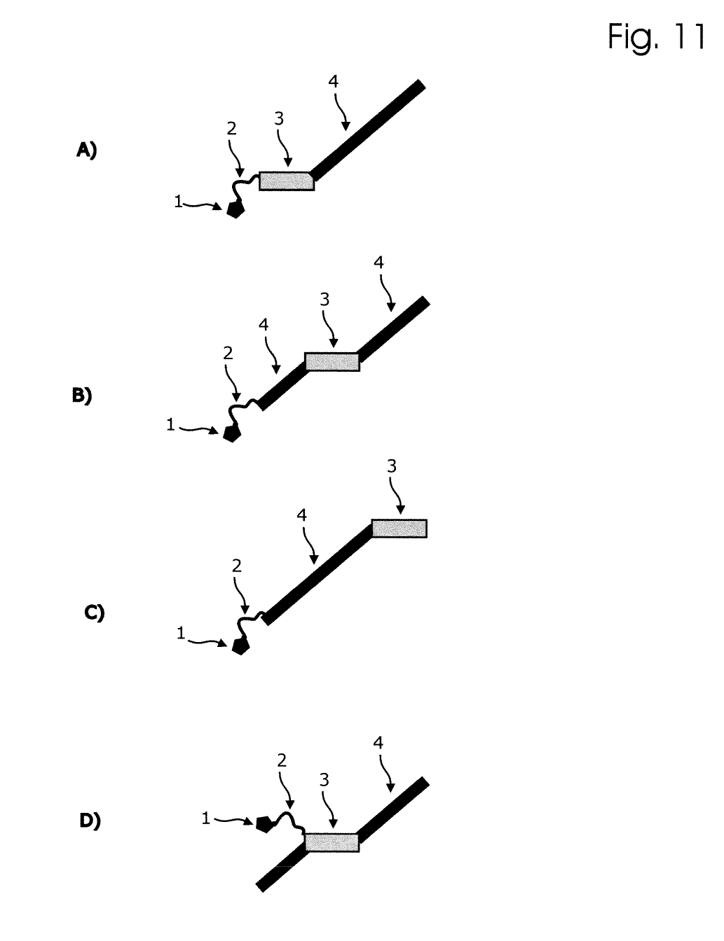

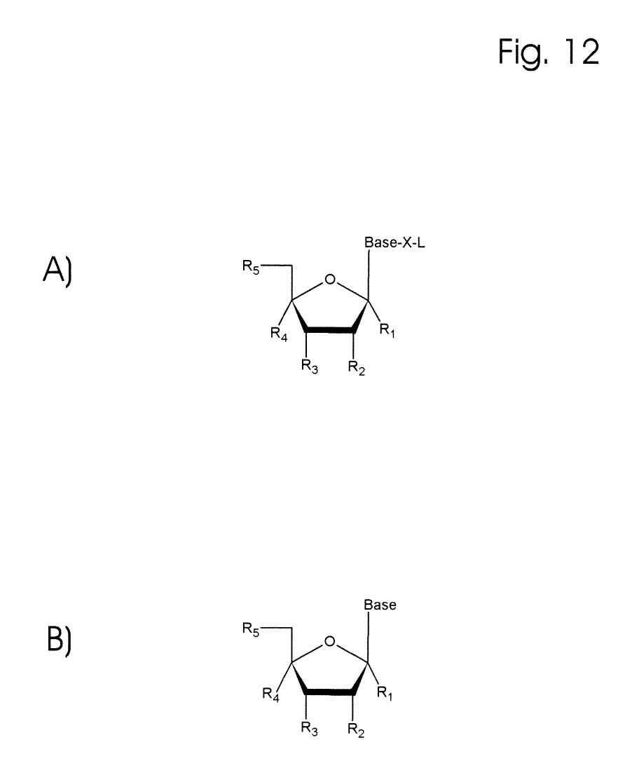

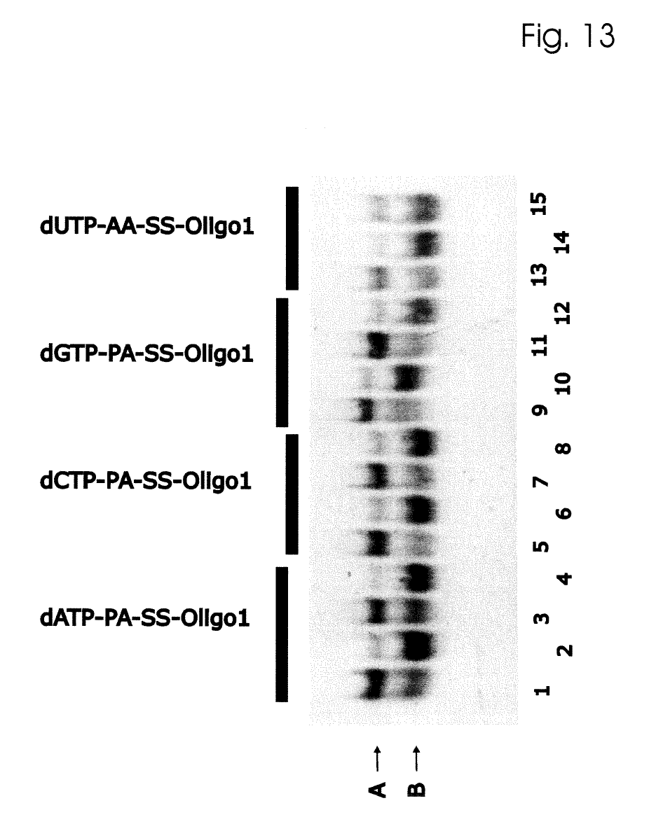

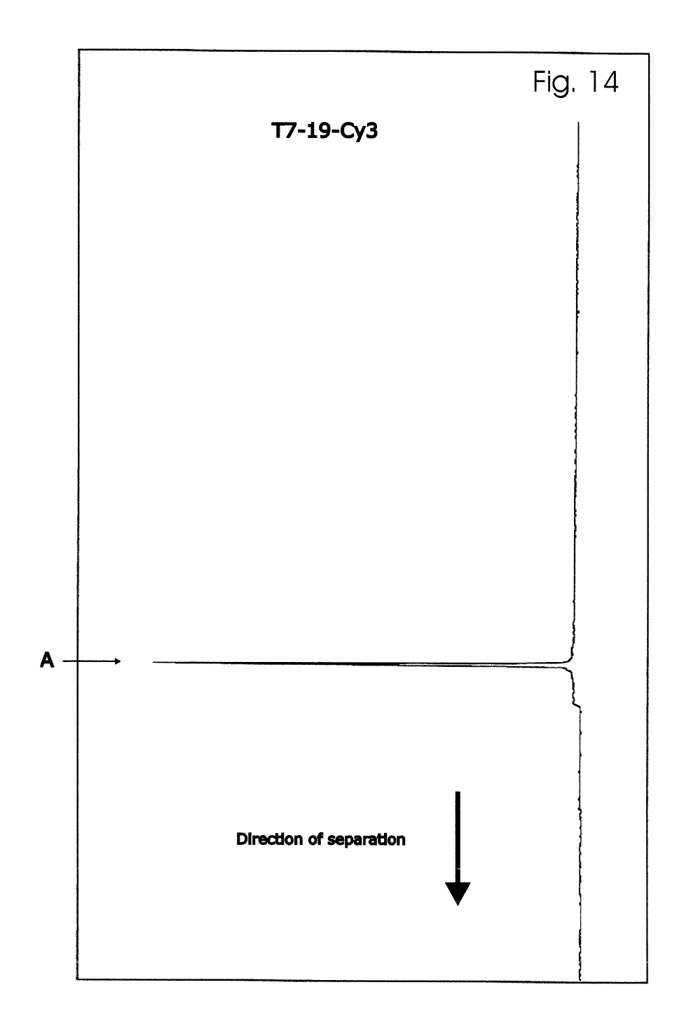

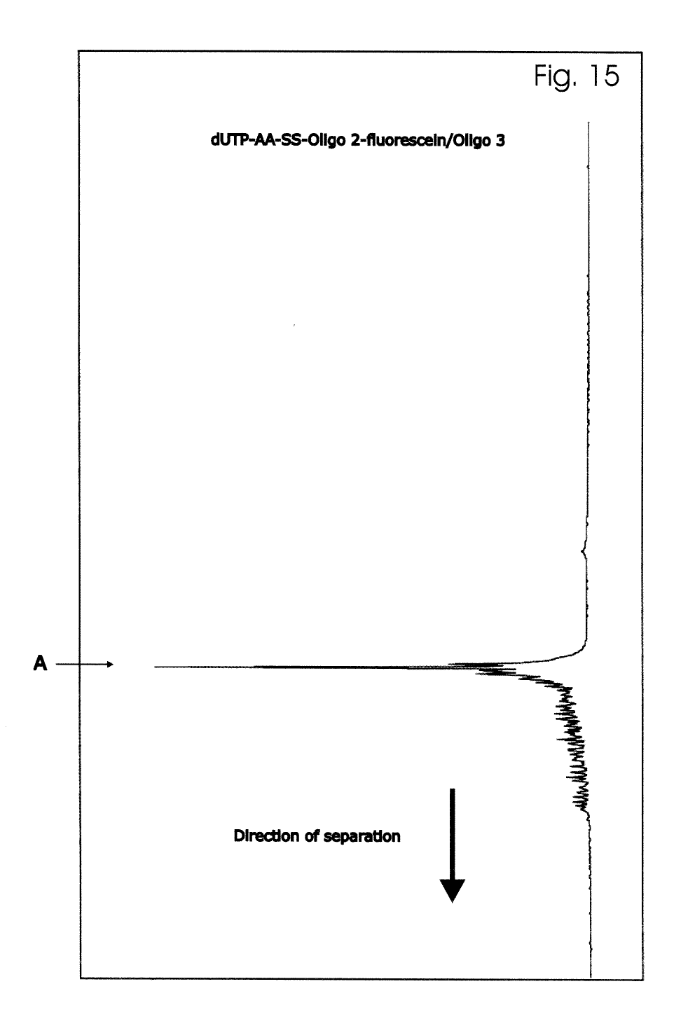

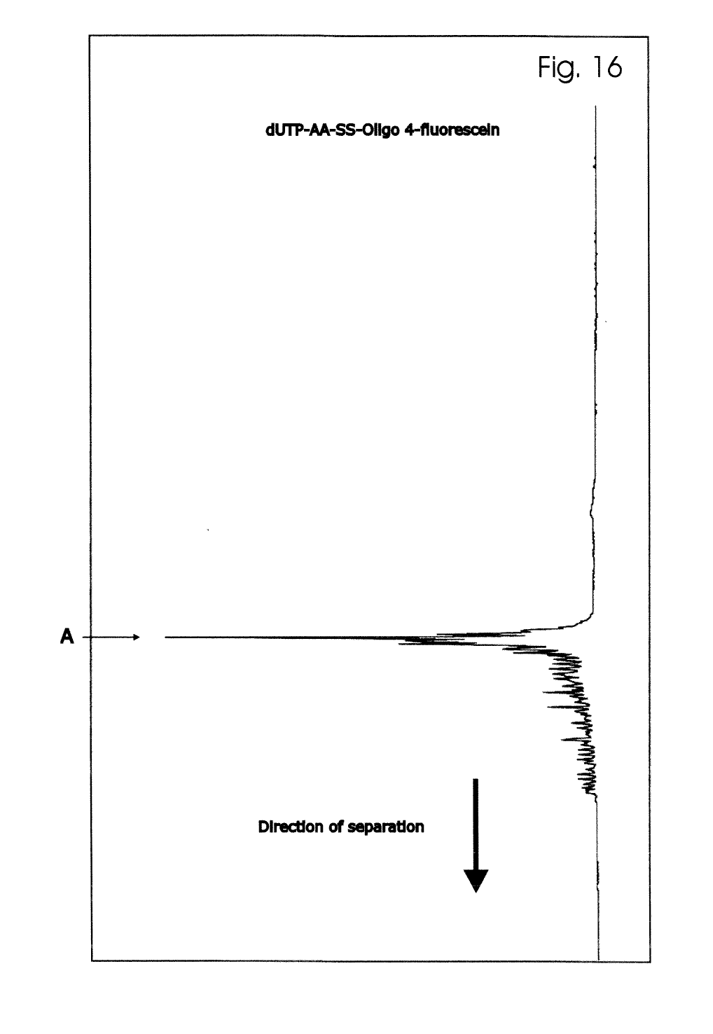

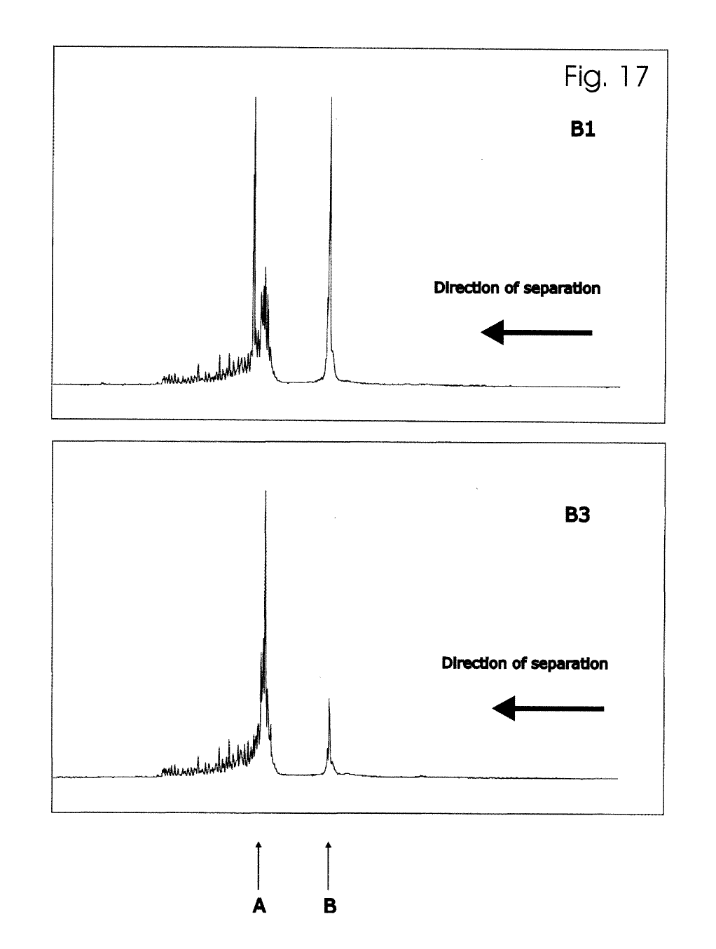

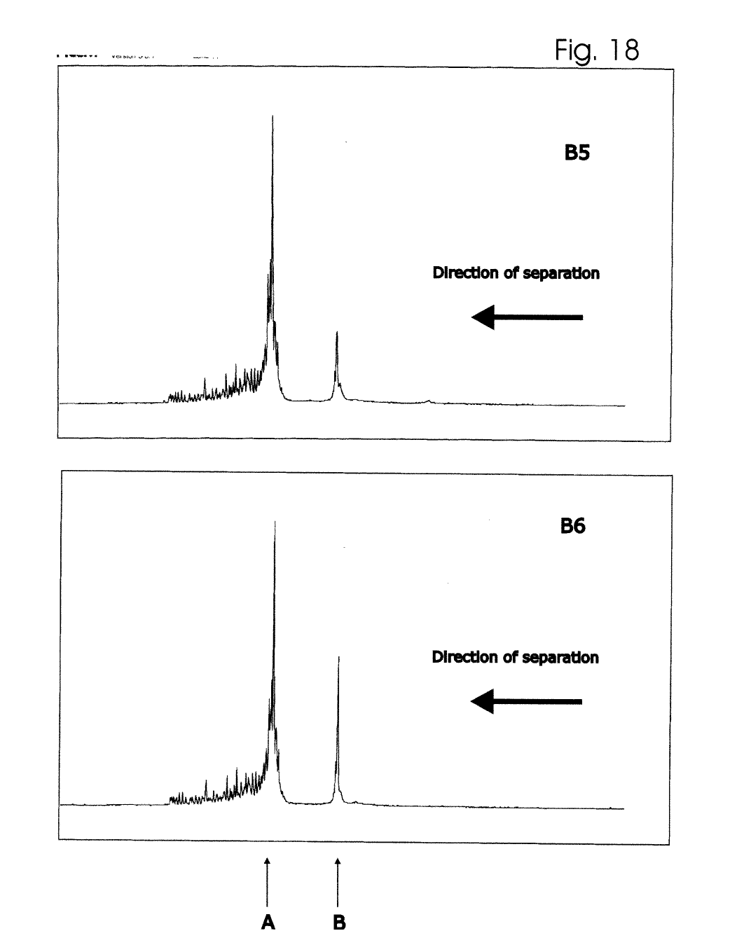

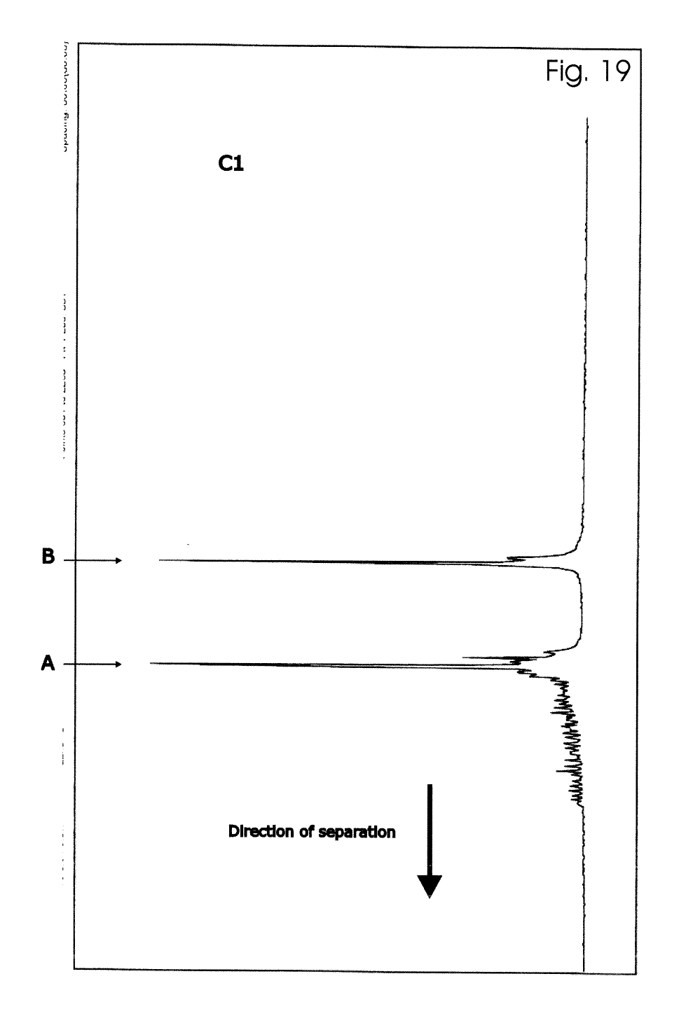

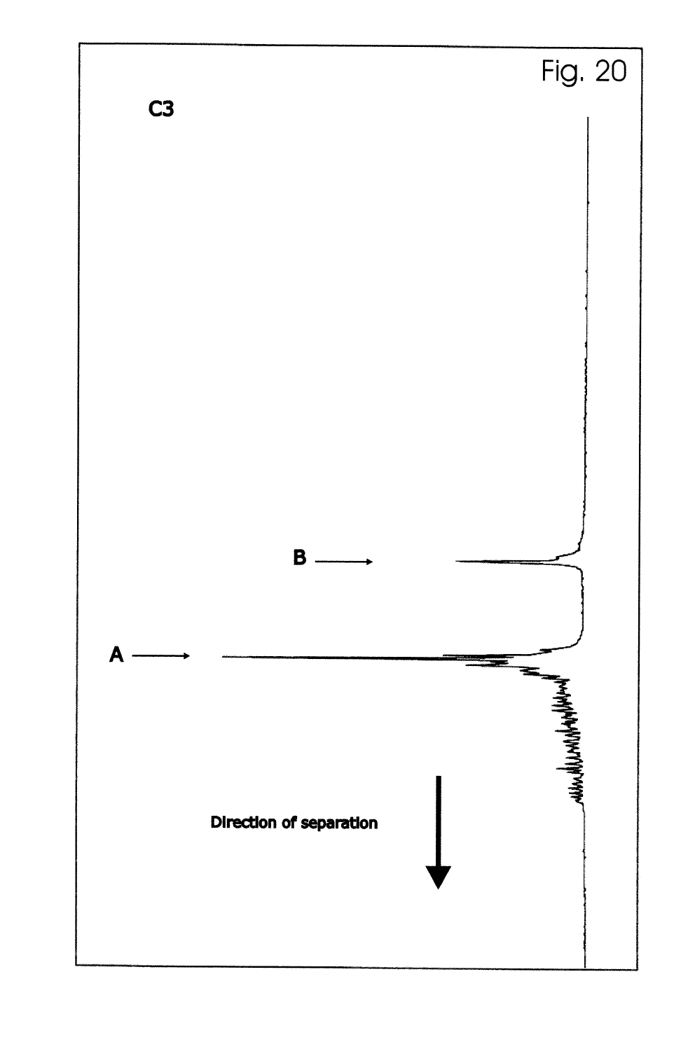

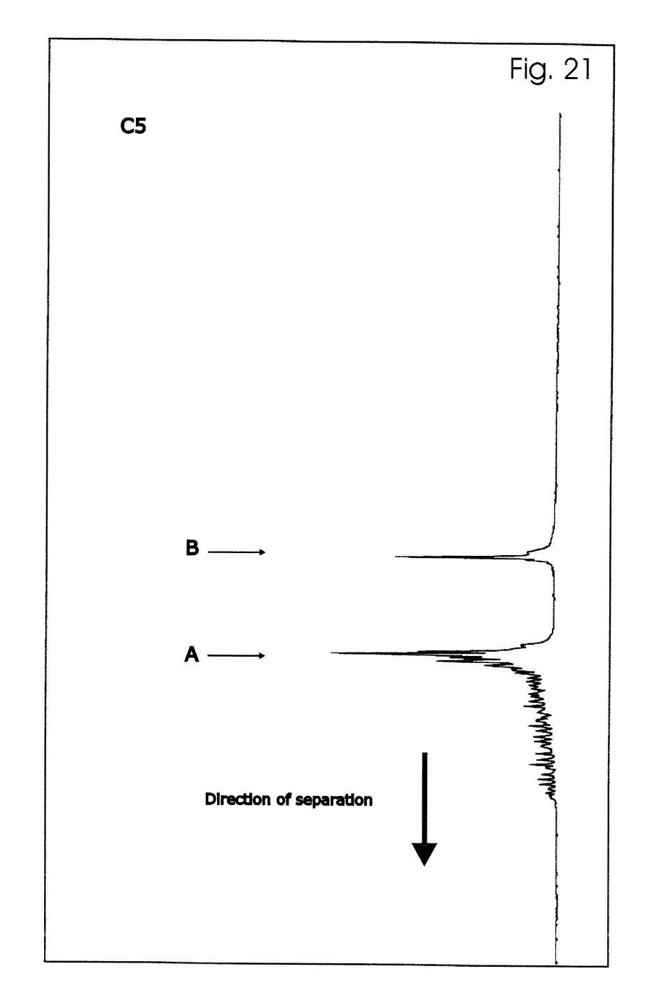

A) Schematic representation of nucleotide conjugates with a uniform nuc-component (1), a linker (2) and a variable segment of the oligonucleotide (3)

B) Schematic representation of nucleotide conjugates with a nuc-component (1), a linker (2) and a uniform oligonucleotide (4)

C) Schematic representation of nucleotide conjugates with a uniform nuc-component (1), a linker (2), a variable segment of the oligonucleotide (3) and a uniform segment of the oligonucleotide (4)

D) Schematic representation of nucleotide conjugates with a uniform nuc-component (1), a linker (2), a uniform segment of the oligonucleotide (4) and a further complementary oligonucleotide (5) with a variable segment (3). The complementary oligonucleotide is hybridized to the uniform oligonucleotide.

FIG. 2

A) Schematic representation of nucleotide conjugates with a nuc-component (1), a linker (2) and a uniform oligonucleotide (3). The oligonucleotide is not complementary to the nucleic acid chain that needs to be analyzed.

B) Schematic representation of extendable template-primer-polymerases: Primer (4), polymerase (5), template (6)

C) Schematic representation of the incorporation event of nucleotide conjugates into the primer by a polymerase

D) Schematic representation of the incorporated nucleotide conjugate together with primer and template prior to removal of the linker and oligonucleotide by cleavage

E) Schematic representation of the incorporated nucleotide after cleavage

F) Schematic representation of a new incorporation event of a second nucleotide conjugate

FIG. 3

A) Schematic representation of four types of nucleotide conjugates used in a sequencing reaction. Each type of nucleotide conjugate is characterized by a uniform nuc-component (corresponding to one of the four bases) and a uniform oligonucleotide specific for each type of nucleotide conjugate.

B) Schematic representation of a sequencing cycle: incubation of primer-polymerase-template complexes with four different types of nucleotide conjugates, incorporation of a nucleotide conjugate complementary to the respective position of the template (nuc-component is incorporated). Subsequent hybridization of a complementary oligonucleotide to incorporated nucleotide conjugates, and detection of the incorporation event, final removal of the linker and of the oligonucleotide with the hybridized labeled oligonucleotide by cleavage.

FIG. 4

A) Schematic representation of nucleotide conjugates with a uniform nuc-component (1), a linker (2), a variable segment of the oligonucleotide (3) and a uniform segment of the oligonucleotide (4)

B) Schematic representation of the incorporation event of nucleotide conjugates into the primer by a polymerase. The nucleotide conjugate is bound to the template through the variable segment of its oligonucleotide (3).

C) Schematic representation of the incorporated nucleotide conjugate together with primer and template prior to removal of the linker and oligonucleotide by cleavage. As the nucleotide conjugate can bind to the template via its variable segment only temporarily, an equilibrium between the bound and free form is observed.

E) Schematic representation of the incorporated nucleotide after cleavage

F) Schematic representation of a new incorporation event of a second nucleotide conjugate

FIG. 5

A) Schematic representation of four populations of nucleotide conjugates, each with a uniform nuc-component (corresponding to the four nucleobases) and oligonucleotides with a variable segment and a uniform segment. The length of the variable segment is (N) bases. Thus, the total number of different oligonucleotides in a population may be calculated from 4̂n. The uniform segments of oligonucleotides can, for example, be specific to a particular population of nucleotide conjugates (for example oligo-A sequence is uniform for all oligonucleotides within the population with the nuc-component dATP, etc.). Preferably, the uniform segments of different nucleotide conjugates are not complementary to one another.

B) Schematic representation of four populations of nucleotide conjugates, each with a uniform nuc-component (corresponding to the four nucleobases) and oligonucleotides with a variable segment and a uniform segment. The variable segment is 3 bases long. Thus, the total number of different oligonucleotides in a population may be calculated as 4̂3=64. The uniform segments of oligonucleotides can, for example, be specific to a particular population of nucleotide conjugates. Preferably, the uniform segments of different nucleotide conjugates are not complementary to one another.

C) Schematic representation of four populations of nucleotide conjugates, each with a uniform nuc-component (corresponding to the four nucleobases) and oligonucleotides with a variable segment and a uniform segment. The variable segment is 4 bases long. Thus, the total number of different oligonucleotides in a population may be calculated as 4̂4=256.

FIG. 6

A) Schematic representation of four populations of nucleotide conjugates, each with a uniform nuc-component (corresponding to the four nucleobases) and oligonucleotides with a variable segment and a uniform segment. The variable segment is 5 bases long. Thus, the total number of different oligonucleotides in a population may be calculated as 4̂5=1024.

B) Schematic representation of four populations of nucleotide conjugates, each with a uniform nuc-component (corresponding to the four nucleobases) and oligonucleotides with a variable segment and a uniform segment. The variable segment is 6 bases long. Thus, the total number of different oligonucleotides in a population may be calculated 4̂6=4096.

FIG. 7

A) Schematic representation of four populations of nucleotide conjugates, each with a uniform nuc-component (corresponding to the four nucleobases, dA, dC, dG, dU) and oligonucleotides with a variable segment and a uniform segment. The variable segment is 4 bases long. Thus, the total number of different oligonucleotides within one population may be calculated as 4̂4=256. The uniform segments of the oligonucleotides can, for example, be specific to a particular population of nucleotide conjugates (for example oligo-A sequence is uniform for all oligonucleotides within the population with the nuc-component dATP, etc.). Preferably, the uniform segments of different nucleotide conjugates are not complementary to one another. Each population of nucleotide conjugates is labeled with a label characteristic for this population (1 to 4), e.g. a fluorescent dye such as Alexa 488, Cy3, Cy5, Cy7.

B) Schematic representation of a sequencing cycle: incubation of primer-polymerase-template complexes with four different types of nucleotide conjugates (see FIG. 7A), incorporation of a nuc-component of the nucleotide conjugate complementary to a respective position of the template (nuc-component is incorporated). Subsequent detection of the incorporation event by measuring the specific signal of the fluorescent dye, final removal of the linker and of the labeled oligonucleotide by cleavage.

FIG. 8

A-C) Schematic representation of nucleotide conjugates with a complementary oligonucleotide. Hybridization can occur at different positions of the complementary oligonucleotide (in the middle (A) at either end (B,C)). Nuc-components (1).

D) Schematic representation of nucleotide conjugates with several complementary oligonucleotides.

E) Schematic representation of nucleotide conjugates with a complementary oligonucleotide that forms a closed loop

F) Schematic representation of nucleotide conjugates with self-complementary segments of the oligonucleotide that form a hairpin-like structure with one another.

FIG. 9

A) Schematic representation of nucleotide conjugates with a nuc-component (1), a linker (2), and a uniform oligonucleotide (3)

B) Schematic representation of nucleotide conjugates with a nuc-component (1), a linker (2), and a uniform oligonucleotide (3), and a complementarily bound oligonucleotide (4)

C) Schematic representation of nucleotide conjugates with a nuc-component (1), a linker (2), and a uniform oligonucleotide (3), and a complementarily bound oligonucleotide (4) that includes a label (e.g. a fluorescent dye or a biotin moiety).

D) Schematic representation of nucleotide conjugates with a nuc-component (1), a linker (2), and a uniform oligonucleotide (6) that includes a label (e.g. a fluorescent dye or a biotin moiety).

E) Schematic representation of nucleotide conjugates with a nuc-component (1), a linker (2), and a uniform oligonucleotide (3) including a label (e.g. a fluorescent dye or a biotin moiety), and a complementarily bound oligonucleotide (8) that also includes a label (e.g. a fluorescent dye or a biotin moiety). The labels can be the same or different. If they are fluorescent dyes, they can form a FRET pair.

F) Schematic representation of nucleotide conjugates with a nuc-component (1), a linker (2), and a long uniform oligonucleotide (9) and several (in this example two) complementarily bound oligonucleotides (10 and 11), where each of the hybridized oligonucleotides includes a label (e.g. a fluorescent dye or a biotin moiety). Both labeled can be the same or different. Both fluorescent dyes can form a FRET pair.

G) Schematic representation of nucleotide conjugates with a nuc-component (1), a linker (2) and a uniform long oligonucleotide (12) and a complementarily bound oligonucleotide (13), wherein the hybridized oligonucleotides include two labels (e.g. a fluorescent dye or a biotin moiety). The labels can be the same or different. If they are fluorescent dyes, they can form a FRET pair. The hybridized oligonucleotide contains a sequence segment which is complementary to oligonucleotide 12 and other flanking sequence segments that are not complementary to oligonucleotide 12. These flanking segments can be complementary to each other.

FIG. 10

Schematic representation of a detection method employing RNase and complementary RNA oligonucleotides

A) Schematic representation of an incorporated nucleotide conjugate

B) Binding of a hybridizing probe consisting of RNA with two fluorescent dyes (RNA probe)

C) Addition of an RNase and cleavage of an RNA-DNA hybrid,

D) Release of cleaved fragments of the RNA probe and regeneration of the binding capacity of the oligonucleotide

FIG. 11

Schematic representation of different positions that may be occupied by the variable segments within the oligonucleotide.

A-C) Schematic representation of nucleotide conjugates with a uniform nuc-component (1), a linker (2), a variable segment of the oligonucleotide (3) as well as a uniform segment of the oligonucleotide (4). The variable segment can be located at the 5′-end of the oligonucleotide (A), in the middle (B) or at the 3′-end (C) of the oligonucleotide. The nuc-component is attached to the 5′-position of the oligonucleotide via the linker.

D) The variable segment is positioned internally and the nuc-component with the linker is also positioned internally, namely at 5′-end of the variable segment.

1. Nucleotide conjugates which comprise the following components: at least one nucleotide component (nuc-component), at least one oligonucleotide, and at least one linker between the nucleotide component and the oligonucleotide.

2. Nucleotide conjugates according to claim 1, where the oligonucleotide comprises at least one single-stranded sequence segment.

3. Nucleotide-conjugates according to claim 1, where the oligonucleotide comprises at least one self-complementary sequence segment.

4. Nucleotide conjugates according to claim 1, where at least one further predominantly complementary oligonucleotide is bound to said oligonucleotide.

5. A reaction mixture or a composition for enzymatic synthesis of nucleic acid chains comprising at least one of the nucleotide conjugates according to claim 1.

6. A method for the enzymatic synthesis of complementary strands of nucleic acid chains, where nucleotide conjugates according to claim 1 are used.

7. A method for labeling nucleic acid chains comprising the following steps:
a) preparation of template-primer complexes capable of extension
b) incubation of these complexes in a reaction solution comprising one or more polymerase types and at least one kind of the nucleotide conjugates according to any claim 1 under conditions which allow for primer extension by at least one nuc-component, where each type of nucleotide conjugate is characteristically labeled and the oligonucleotide of the nucleotide conjugates is complementary to the nucleic acid chain provided in (A) over at least 3 bases and at most 10 bases.

8. A method for sequencing nucleic acid chains comprising the following steps:
a) preparation of at least one population of extendable template-primer complexes (NAC-primer complexes)
b) incubation of at least one type of nucleotide conjugate according to claim 1 and at least one type of polymerase together with the NAC-primer complexes provided in step (a) under conditions which permit incorporation of complementary nuc-components of the nucleotide conjugates, where each type of nucleotide conjugate bears a particular characteristic label.
c) separation of the unincorporated nucleotide conjugates from the NAC primer complexes
d) detection of the signals of the nucleotide conjugates incorporated into NAC-primer complexes
e) cleavage of the linker component as well as of the marker component and oligonucleotide component from the nucleotide conjugates incorporated into the NAC-primer complexes
f) washing of the NAC-primer complexes
repetition of steps (b) through (f) if required.

9. A method for sequencing nucleic acid chains comprising the following steps:
a) preparation of at least one population of extendable template-primer complexes (NAC-primer complexes)
b) incubation of at least one type of nucleotide conjugate according to claim 1 and at least one type of polymerase together with the NAC-primer complexes provided in step (a) under conditions which permit incorporation of complementary nuc-components of the nucleotide conjugates, where the oligonucleotide of the nucleotide conjugates is not complementary to the under nucleic acid chains prepared in step (a), and where each type of nucleotide conjugate bears a particular characteristic label
c) separation of the unincorporated nucleotide conjugates from the NAC-primer complexes
d) detection of the signals of the nucleotide conjugates incorporated into the NAC primer complexes
e) cleavage of the linker component as well as of the marker component and oligonucleotide component from the nucleotide conjugates incorporated into the NAC-primer complexes
f) washing of the NAC-primer complexes
repetition of steps (b) through (f) if required.

10. A method for sequencing nucleic acid chains, comprising the following steps:
a) preparation of at least one population of extendable template-primer complexes (NAC-primer complexes)
b) incubation of at least one type of nucleotide conjugate according to claim 1 and at least one type of polymerase together with the NAC-primer complexes provided in step (a) under conditions which permit incorporation of complementary nue-components of the nucleotide conjugates, where the oligonucleotide of the nucleotide conjugates is complementary to the nucleic acid chains prepared in step (a) over at least 3 bases and at most 10 bases, and where each type of nucleotide conjugate bears a particular characteristic label
c) separation of the unincorporated nucleotide conjugates from the NAC primer complexes
d) detection of the signals of the nucleotide conjugates incorporated into the NAC primer complexes
e) cleavage of the linker component as well as of the marker component and oligonucleotide component from the nucleotide incorporated conjugates into the NAC-primer complexes
f) washing of the NAC-primer complexes
repetition of steps (b) through (f) if required.


SEQUENCE LISTING (19 sequences)

SEQ ID NO: 1 - DNA, 19 bp, Artificial Sequence
  Description of Artificial Sequence Synthetic Oligo 1 sequence
taatacgactcactatagg

SEQ ID NO: 2 - DNA, 33 bp, Artificial Sequence
  Description of Artificial Sequence Synthetic Oligo 2 fluorescein sequence
cgtattaccgcggctgctggcacaaaaaaaaaa

SEQ ID NO: 3 - DNA, 35 bp, Artificial Sequence
  Description of Artificial Sequence Synthetic Oligo 4 sequence
cgtattaccgcggctgctgtaatacaaaaaaaaaa

SEQ ID NO: 4 - DNA, 23 bp, Artificial Sequence
  Description of Artificial Sequence Synthetic Oligo 3 sequence
gtgccagcagccgcggtaatacg

SEQ ID NO: 5 - DNA, 19 bp, Artificial Sequence
  Description of Artificial Sequence Synthetic primer T7 19
taatacgactcactatagg

SEQ ID NO: 6 - DNA, 39 bp, Artificial Sequence
  Description of Artificial Sequence Synthetic Oligo 5 sequence
nnnnnncgtattaccgcggctgctggcacaaaaaaaaaa

SEQ ID NO: 7 - DNA, 39 bp, Artificial Sequence
  Description of Artificial Sequence Synthetic Oligo 6 sequence
ttttttttttcgtattaccgcggctgctggcacnnnnnn

SEQ ID NO: 8 - DNA, 68 bp, Artificial Sequence
  Description of Artificial Sequence Synthetic template 1 oligonucleotide
gttttcccagtcacgacgggaggtgccagcagccgcggtaatacgaccacctatagtgagtcgtatta

SEQ ID NO: 9 - DNA, 25 bp, Artificial Sequence
  Description of Artificial Sequence Synthetic template 2 oligonucleotide
aaaaaacctatagtgagtcgtatta

SEQ ID NO: 10 - DNA, 20 bp, Artificial Sequence
  Description of Artificial Sequence Synthetic template 3 oligonucleotide
acctatagtgagtcgtatta

SEQ ID NO: 11 - DNA, 24 bp, Artificial Sequence
  Description of Artificial Sequence Synthetic template 4 oligonucleotide
tttttcctatagtgagtcgtatta

SEQ ID NO: 12 - DNA, 27 bp, Artificial Sequence
  Description of Artificial Sequence Synthetic template 5 oligonucleotide
cgctttgtcctatagtgagtcgtatta

SEQ ID NO: 13 - DNA, 23 bp, Artificial Sequence
  Description of Artificial Sequence Synthetic template 6 oligonucleotide
agggcctatagtgagtcgtatta

SEQ ID NO: 14 - DNA, 20 bp, Artificial Sequence
  Description of Artificial Sequence Synthetic template 7 oligonucleotide
gcctatagtgagtcgtatta

SEQ ID NO: 15 - DNA, 24 bp, Artificial Sequence
  Description of Artificial Sequence Synthetic template 8 oligonucleotide
actctcctatagtgagtcgtatta

SEQ ID NO: 16 - DNA, 10 bp, Artificial Sequence
  Description of Artificial Sequence Synthetic oligonucleotide
aaaaaaaaaa

SEQ ID NO: 17 - DNA, 11 bp, Artificial Sequence
  Description of Artificial Sequence Synthetic oligonucleotide
aaaaaaaaaaa

SEQ ID NO: 18 - DNA, 10 bp, Artificial Sequence
  Description of Artificial Sequence Synthetic oligonucleotide
tttttttttt

SEQ ID NO: 19 - DNA, 23 bp, Artificial Sequence
  Description of Artificial Sequence Synthetic oligonucleotide
cgtattaccgcggctgctggcac